SEQUENCE LISTING (66 sequences)

SEQ ID NO: 1 - DNA, 1791 bp, Aspergillus nidulans
  gene 1..1791: note = A.nidulans AN4663 gene encoding histidine methyltransferase
  source 1..1791: mol_type = genomic DNA
atggcgccattcaggagcatctatgagaaagatgctaccaagaagctggtagtcggagcagccttattggtgcttgcggctttctactcctacgtcttcctgttgacgctggccccagtctatgggtcaactccgtcgcacatcttccacggctacggcgtcggcatcgcaggcgtagcaggctggttctccaaggacatcgtcgaccgggtgtctgggcgcaaggcgatctacgctatccctgtgctggccttctttctgcctgttgtccagtactttgtctcccagcagagctcggcgctgggaaatcctgctggcccaatcttcaccgaagtactggcgttatacccgctggttctgctctctgtggcttgtgcgggcaaattggtgcaggcaggcttgaacctccaacgacatggcgatctggttgcggagcatatccctctactgggatcgtatgtcatctactctgccggcgagcacttgatcaaggccttcttgtcaaggttcattgggtcgactgtgctgctcagccgcgcgggtcttcagatcttgattgcaatcttttacgcggcagccgttccctctaaggctctccttctagctatccctgcttttcttttctctgtcacttcaaacacgcacctgccgttgggacacacgactacagccctcaacaacatcatcgccgatgatggattcgctcttgtcgcccgtcaggattccacaacggggtatatctccgtcctcgacaatcttgaggatggcttccgcgtcatgagatgcgaccacagcctccttggtggccaatggatcaaaaagcgccccaactacacccctcccgctgtcaaggaccctatctacgctgtgtttactatgctggaggccgtgagactcgtcgagacagcgcacggtataccacgtgctgacgcaggaagtaacgcgctcgtcatgtaaacttttcccccatggcttccgctgacagaagacactaacgtttcacagtgggctcggaattggcacaacccctggcgcgctgataagccacggaatcgacacgaccatcgtcgaaatcgacccggttgtccacaagtatgctctgcagtacttcgatctccccgaaaaccacacccctatcatcgaagacgcgcgggccttcgtccagcgcagccgaaacgcaccccaacccaagcagtacgactacattgtccacgacgtctttactggtggtgcagaacctgttgagctcttcacgtatgagttcattagtggtctacacgcactcttgaaggatgacggcgttattgccattgtatgcctcccccaacacctcaaaaccttctaaatatacgcaggataactaaccgactgaagaattatgccggcgacatctctctctattccacggccctctccatccggaccataaaatccatcttcccaacctgccgtctcttccgtgaagccgcggccccggaaatcgggcccgacttcacaaacatggtcatcttctgcacgaaatcccgtggtgcgcctatcaccttccgcgaccccgttcccgaggacttcctgggcagccgcttccggtcccgttacctggtaccaaagcatgaggtcgacgcggcgcaatttgataatgtcgggttagaggacggtcctcaaggtcatgggcgcagggttctagtggataaagaggttggtcgcttacataagtatcaggatcgctcggcgctggagcactgggggattatgaggactgttttgccggatagggtctgggaggggtggtag

SEQ ID NO: 2 - protein, 558 aa, A. nudulans
  PEPTIDE 1..558: note = A. nudulans AN4663 methyltransferase
  DOMAIN 220..558: note = Predicted catalytic domain of AN4663
MAPFRSIYEKDATKKLVVGAALLVLAAFYSYVFLLTLAPVYGSTPSHIFHGYGVGIAGVAGWFSKDIVDRVSGRKAIYAIPVLAFFLPVVQYFVSQQSSALGNPAGPIFTEVLALYPLVLLSVACAGKLVQAGLNLQRHGDLVAEHIPLLGSYVIYSAGEHLIKAFLSRFIGSTVLLSRAGLQILIAIFYAAAVPSKALLLAIPAFLFSVTSNTHLPLGHTTTALNNIIADDGFALVARQDSTTGYISVLDNLEDGFRVMRCDHSLLGGQWIKKRPNYTPPAVKDPIYAVFTMLEAVRLVETAHGIPRADAGSNALVIGLGIGTTPGALISHGIDTTIVEIDPVVHKYALQYFDLPENHTPIIEDARAFVQRSRNAPQPKQYDYIVHDVFTGGAEPVELFTYEFISGLHALLKDDGVIAINYAGDISLYSTALSIRTIKSIFPTCRLFREAAAPEIGPDFTNMVIFCTKSRGAPITFRDPVPEDFLGSRFRSRYLVPKHEVDAAQFDNVGLEDGPQGHGRRVLVDKEVGRLHKYQDRSALEHWGIMRTVLPDRVWEGW

SEQ ID NO: 3 - DNA, 1771 bp, Aspergullis fumigatus
  gene 1..1771: note = Gene encoding A. fumigatus methyltransferase
  source 1..1771: mol_type = genomic DNA
atgtcttctcccggaatgttccagcaagtcaagcttgaaaagcttctgccaggtgtagcattgctgttcctcactgctttctattcccctgtctttctcttgacactgtctccggtttttggctctgcgccttcccatatctttcacgggtacggagtagctgtcagtgccgctgcaggctggttcttgaaggacgtgatccatcggctgtctggccgcagagcagtatatttggtccccgttctcgcattctgggtatcgaccatccagtacttcatgtttcaccagagctcatctttcgggaatccaactggcccgctaattattgacatatttaccttctaccctctggtactcctttctgtggcttgcgccgggaaactattgcaggcgggcctgaacatgagccagcatggagaccttatgacagagcatattccccttctcgggtcctatgttatctactccgcaggggaatatttttcaaaagcgtttatctcgaagttcattggcttgaccttcttcctgagccgtggcggccttcaattactgattgcgagcttgtattcggctaccatcccatctaaatggcttgtattagcggttccatctattctcttctctctcacgtttaatgtgcacatgcccttcggccgcacaactgctgctctgaactctgctattcaagtggaaggatatgaacttctcgctagacaggattcttcaaccgggtacatatccgttttggagaacttggaagatggcttccgagtgatgcgatgtgatcatagtctccttggcggtcaatggacaaagatgccgcgcaattaccatcccgaggtcaaggaccccatttacgctgtgtttgctatgctcgaagccatccgtttgatcgaaaccgaccacggcgaggcgcgtccggacgccaacagcagggctcttgtcatgtaaggagccctatcgacgccaatttgtggaggtaaggtagtcagctaactggactttcttagtggacttggggttgggaccacccccgctgcgttgattacgcacggcatcgagaccacaatcgtggagatagatccggttgtgcataaatttgccatggaatacttccacctaccttccagtcacattgcagctattgaagacgcttcagtgtttgtgaagcgtgcccaggaatccccaaaccctgctcaatacgactacattgtccatgacgtcttcaccggcggggcggagccagtcgagctcttcacaatcgaattcatcaagggtctatatgacctattgaaagaggatggtgttattgccattgtaagaccctcccctccgttactaactccgtcacctcatgactgaacgatatctatagaactacgccggtgatatctcactttacccagccgcactcacggtgcgcacaattaaggcagtcttcccgtcgtgcaggatcttccgcgaagacgcagccgcggatgccaagttcgatttcaccaacatggtaatcttctgcaagaagagcaacacaccccttcgcttcagagatccagtcaaagccgatttcctgggaagcaagtctcgtcaacactaccttgtgcccaagcacgaggtggacccagccaaattcgaaaaggttgagaagggtggaaggcgtgttctggttgcaaaggaaacgggccggcttcataagtaccaggatcgtggtgcaattgcccattgggagatcatgagaacagtcatgccggatgcggtatgggagaattggtaa

SEQ ID NO: 4 - protein, 549 aa, A. fumigatus
  REGION 1..549: note = peptide - A. fumigatus methyltransferase
  REGION 302..497: note = domain - Predicted catalytic domain of A. fumigatus methyltransferase
MSSPGMFQQVKLEKLLPGVALLFLTAFYSPVFLLTLSPVFGSAPSHIFHGYGVAVSAAAGWFLKDVIHRLSGRRAVYLVPVLAFWVSTIQYFMFHQSSSFGNPTGPLIIDIFTFYPLVLLSVACAGKLLQAGLNMSQHGDLMTEHIPLLGSYVIYSAGEYFSKAFISKFIGLTFFLSRGGLQLLIASLYSATIPSKWLVLAVPSILFSLTFNVHMPFGRTTAALNSAIQVEGYELLARQDSSTGYISVLENLEDGFRVMRCDHSLLGGQWTKMPRNYHPEVKDPIYAVFAMLEAIRLIETDHGEARPDANSRALVIGLGVGTTPAALITHGIETTIVEIDPVVHKFAMEYFHLPSSHIAAIEDASVFVKRAQESPNPAQYDYIVHDVFTGGAEPVELFTIEFIKGLYDLLKEDGVIAINYAGDISLYPAALTVRTIKAVFPSCRIFREDAAADAKFDFTNMVIFCKKSNTPLRFRDPVKADFLGSKSRQHYLVPKHEVDPAKFEKVEKGGRRVLVAKETGRLHKYQDRGAIAHWEIMRTVMPDAVWENW

SEQ ID NO: 5 - DNA, 1981 bp, N. crassa
  gene 1..1981: note = Gene encoding N. crassa methyltransferase
  source 1..1981: mol_type = genomic DNA
atgccccccaaagcaacaaaaaggggcaaggcctccaaggccaccaccaccaccaccaccacccccgaagaatcagaaccaaaaacaacagcaacatccgcccctgacctcttcaccaaatccaaggaagcctacgagcaagaactcaaagacctcgccaaaaaggcccgctcccaaaagggctactgggccggccagttcgccatctacacccacaccttcatcctgctcgctctcatcgccatcgcctccaacgtctcccagctcgttctctcgcccgtctacggctccatccccgcggcgatatggcactccaagctagtcatggccggctgtttcaccggctgggcctccaacatcttcttggatcgcgctttgcccattgagccagtcgagctgctgcctctgatcgccatgtacattccgcccgctcagtacctccttttcaaggccagcggctacatgggcgcttactatggcccattggtcacggagctgttgacgctattccccctgattaccgtctcctcggcctgtgtcgcgacaaacctggagtatgccgactttggaccgctgcccaaagccattgctgaggcggtgcctggcctgggctccttcggcttcttcaaagtggccgagaagttgagcggcgactttattgtgcagaacatcggcaagaacttttacatgaccaggatcggactggagggcttgctggctgcttcctacacgaccattgcgccgtccaagttcttgttgttgggggtgcccgcgttgttgcacaccacgcttttgaacccgcatctggcctcgcctatggcgctttctaggttgaacagcgggctggagaaggagggatggcatgtgctggaccgccgcgactcgatcacgggatatatctcggtggtggacagtttgaatgatgggttccgagtgttgaggtgtgaccacagtctgcttggtggcgagtgggtcaagttcaaggacactcctcggttcaaggggaaccaggttgctgagcctatctatggtgtctttgccatgttggaggccgtgaggttggtccaagtgcctaacccggttcctgacaacgaggcgaaggctttggtaatgtaagttccccccgttctagcatctcgagatggaaaactaaacatattccagcggcctcggcatcggcaccacccccgccgccctcgttgcccacggcatcaacaccaccgttgttgaaatcgatcctgtcgtccacgagttcgcctccaagtacttccagcttccgtccaaccacaccgccgtcatcgaagacgccgtaagctacaccgaccgtctcgtcaacgaaactcaaggtgtcggtcaatatgaatacatcatccacgacgtctttaccggcggcgctgagcccgtcgccctcttcacttatgaattcctccaaaacttgcactcccttctcaagccaaacggcgtcatcgccatcgtacgtcacccctcccctcctctttctcttcctataaccacaactaacatcacaacgcagaactacgcaggcgacttctcccaccccccacccctcctcatcaccaacaccatccgctccgtctttggttcttcctcatgccgcatcttccgcgaacaccctcgcgacgaagcccaagccgccgcccacaacggccgcgatttcacgaatatggtctttttctgcaccaaggtttctactcccactcagggaattactttccgcaacccaaccccccgtgacctcctcaacagtcccagccgcgaagccttccttctccccaagtttgaggtgacagacgacgatctcctcagagctgtcaatggcagtgtagaagaggctaagaagctggggattttgcgaaagaatgagacagcgacgttggaaaagtggcagacggaaagcgcgctgggccattgggagattatgaggggggtgttgcctgaggttgtgtgggttaattggtag

SEQ ID NO: 6 - protein, 622 aa, N. crassa
  REGION 1..622: note = peptide - N. crassa methyltransferase
  REGION 353..497: note = domain - Predicted catalytic domain of N. crassa methyltransferase
MPPKATKRGKASKATTTTTTTPEESEPKTTATSAPDLFTKSKEAYEQELKDLAKKARSQKGYWAGQFAIYTHTFILLALIAIASNVSQLVLSPVYGSIPAAIWHSKLVMAGCFTGWASNIFLDRALPIEPVELLPLIAMYIPPAQYLLFKASGYMGAYYGPLVTELLTLFPLITVSSACVATNLEYADFGPLPKAIAEAVPGLGSFGFFKVAEKLSGDFIVQNIGKNFYMTRIGLEGLLAASYTTIAPSKFLLLGVPALLHTTLLNPHLASPMALSRLNSGLEKEGWHVLDRRDSITGYISVVDSLNDGFRVLRCDHSLLGGEWVKFKDTPRFKGNQVAEPIYGVFAMLEAVRLVQVPNPVPDNEAKALVIGLGIGTTPAALVAHGINTTVVEIDPVVHEFASKYFQLPSNHTAVIEDAVSYTDRLVNETQGVGQYEYIIHDVFTGGAEPVALFTYEFLQNLHSLLKPNGVIAINYAGDFSHPPPLLITNTIRSVFGSSSCRIFREHPRDEAQAAAHNGRDFTNMVFFCTKVSTPTQGITFRNPTPRDLLNSPSREAFLLPKFEVTDDDLLRAVNGSVEEAKKLGILRKNETATLEKWQTESALGHWEIMRGVLPEVVWVNW

SEQ ID NO: 7 - DNA, 2264 bp, N. lepideus
  gene 1..2264: note = Gene endcoding N. lepideus methyltransferase
  source 1..2264: mol_type = genomic DNA
atgcctgacgacaaaatacttggactggtctccaaggagagggtcctgaagaccatcaaaggaggcatcctctccatgtctctgacaacgtccctctccctcatcatcttcacctaccaacgagctctcgaaccaatatatggctccgttcccaccagtctctatctctggtatgttgttgtgggtgccggctcgctgggggtctttgctcccgtcttcaatttatggagcgcgacaggactggtaggtgctctgctgtcagtggtgccgtatagcacgtattgggttggtgtgtattctgcccggtatcacgatcctatctacggtcctttggccacgcacctagctgtgttggcaccaatcatctacctcggcgcgaacatacgaatcacgatggaagtgagttatgtcaggctcagggttatcctaccctcctggctgccaaagccttccttctcttcattattgataccatttggatgcctggcgtccatagactgataagcctggttctcctctcattacaaatccgctctctgcattcttctccttcactggtttctttggactagtcaagaactctgcatatttctcgtggctcactttcctcaactatattccaaatgaattcatagtgagcaacatttaacgagatgttacaatgccatccaattaccgatcactacagctctacgctctctctgtaatcggagtatttttatggggcatatccgaccggatcacccaggcttctgctcctcccgtaaaggataagagcgagaagactgggaagtcagataaagacatgaagtccaactcgaaggaagctgagaaagcaaaggaaaagccgaaagcgaccactggcctcagcttccgacagatgtacctctttccattaatcgttgtactgcctttcctccagtcctacatacgacctcccacactactacgtccccttatggaggctcatacccactcagcttatccgcttcgaatcctctcatctacgcaatcgaccacgggcgtcatagtcgtcggcgaatccctcgctgtaagcgcggcgttcttcgcaagcgaaataccagaaaatcacattgagagcatcaggtacctcagagcaggccattcactcttgggcggtgtgtggatatccgacaaggtggcgtatatagaaggtatacaacgcggagatgtcccggtagatgccaatgatcagccccttggtgattcaatttatgcagcgtttgtattgcaagaaatctctcggttggccaagagacagagtgccgtggctgaggaacagaatgccctaattatgtgcgcggatcctcttacacgtgcatgtcatttctgatgcacgcccttagaggtctaggcattggcgtggcagcgtcttcattcatgcgacacggcattaacacgactattgtcgagatcgaccccgctgtatacgatgccgctcgatattacttcggtctgcccaaagccaaccctggccaggtctacctgcaagacgctaagcgctgggtaaagcgtcaatcctccctgccggaaggacagagacagttatatgacatcgtcatacatgattgcttctccggaggcggcgttcctgctcaaatcttcacgatggaattttgggatgggttgaagaagctgatgaagcctgatggggtcgttgccgtggtacgacagacctttctatgtgaccggcgggacactcaccaagctatgtactagaactttgctgggaaattaactgggaagccatcgcgcgcaattcttgcgacgctagagaggagctttaagacctgtcgcgcgttccatgactcaatgtcggaagtctctgagaaacagatgaagacggagttcctgaacatcgtgagttgcctgtgtatacaagttacctgtttctctaaggatgagttgtcccaggtatttttttgtactccctcgtcggagccactcacattccgggagcccatcgcgcgcgattatcatcattcgtatttgcgtgaacatgtttttacgacgccgcgagagatcgatctagcattgatcaaagagggtgtgtctgtggaggacgaagctgagtatgttctgacagacaagaagaacaaactggagaagtggcaggaggaaggcgcgttggagcattggaagggtgagcgtttcatgttcgctctaaacgagactgtgctgatggatcacgctccctagtgatgcgggaggtgttgcctgacatcttttgggaaacgtactag

SEQ ID NO: 8 - protein, 632 aa, N. lepideus
  REGION 1..632: note = peptide - N. lepideus methyltransferase
  REGION 385..496: note = domain - Predicted catalytic domain of N. lepideus methyltransferase
MPDDKILGLVSKERVLKTIKGGILSMSLTTSLSLIIFTYQRALEPIYGSVPTSLYLWYVVVGAGSLGVFAPVFNLWSATGLVGALLSVVPYSTYWVGVYSARYHDPIYGPLATHLAVLAPIIYLGANIRITMETDKPGSPLITNPLSAFFSFTGFFGLVKNSAYFSWLTFLNYIPNEFILYALSVIGVFLWGISDRITQASAPPVKDKSEKTGKSDKDMKSNSKEAEKAKEKPKATTGLSFRQMYLFPLIVVLPFLQSYIRPPTLLRPLMEAHTHSAYPLRILSSTQSTTGVIVVGESLAVSAAFFASEIPENHIESIRYLRAGHSLLGGVWISDKVAYIEGIQRGDVPVDANDQPLGDSIYAAFVLQEISRLAKRQSAVAEEQNALIIGLGIGVAASSFMRHGINTTIVEIDPAVYDAARYYFGLPKANPGQVYLQDAKRWVKRQSSLPEGQRQLYDIVIHDCFSGGGVPAQIFTMEFWDGLKKLMKPDGVVAVNFAGKLTGKPSRAILATLERSFKTCRAFHDSMSEVSEKQMKTEFLNIVFFCTPSSEPLTFREPIARDYHHSYLREHVFTTPREIDLALIKEGVSVEDEAEYVLTDKKNKLEKWQEEGALEHWKVMREVLPDIFWETY

SEQ ID NO: 9 - DNA, 442 bp, Saccharomyces cerevisiae
  regulatory 1..442: note = promoter - gal1 promoter; regulatory_class = promoter
  source 1..442: mol_type = genomic DNA
cggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggag

SEQ ID NO: 10 - DNA, 936 bp, K. phaffii
  regulatory 1..936: note = promoter - AOX promoter; regulatory_class = promoter
  source 1..936: mol_type = genomic DNA
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaa

SEQ ID NO: 11 - DNA, 18 bp, E. coli
  regulatory 1..18: note = promoter - T7 promoter; regulatory_class = promoter
  source 1..18: mol_type = genomic DNA
taatacgactcactatag

SEQ ID NO: 12 - DNA, 271 bp, E. coli
  regulatory 1..271: note = promoter - Tetracycline inducible promoters (tetracycline response element); regulatory_class = promoter
  source 1..271: mol_type = genomic DNA
tccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagaga

SEQ ID NO: 13 - DNA, 39 bp, human cytomegalovirus
  regulatory 1..39: note = promoter - TET- responsible promoter: CMV (Cytomegalovirus minimal promoter); regulatory_class = promoter
  source 1..39: mol_type = genomic DNA
ggtaggcgtgtacggtgggaggcctatataagcagagct

SEQ ID NO: 14 - DNA, 747 bp, Lentinus similis
  gene 1..747: note = gene encoding Lentinus similis LPMO
  source 1..747: mol_type = genomic DNA
cataccctcgtctggggcgtatgggtcaacggcgttgatcaaggagatgggaggaacatctacatcaggtctcccccgaacaataaccccgtcaagaacctgacttcgccggatatgacgtgcaatgtcgacaatcgcgttgtgcccaagagcgtgccagtcaacgctggtgatacgcttactttcgaatggtaccacaacacacgagacgacgatatcatcgcatcttctcatcacggacccatcgccgtatacatcgcacctgccgcttcgaacggtcaagggaacgtttgggtgaagctgttcgaggacgcctacaacgtcaccaatagcacttgggctgtcgatcgtctcatcactgctcatggacaacactccgttgtggtcccccatgttgcacctggagactacctatttagagctgagatcattgcgctacatgaggcagattctctgtactcccagaaccctatccgtggcgcgcaattttatatctcgtgcgctcaaatcaccatcaactcttccgatgactcgacacctctccccgctggtgttcccttccccggtgcatacaccgactccacacccggtattcaattcaatatctacactacccccgcgacaagctacgttgctccccctcccagcgtatggtctggagcgttgggtgggtcgattgctcaggtcggagatgcttctctcgaggaaaacctctacttccagggccatcatcaccatcaccactaa

SEQ ID NO: 15 - protein, 248 aa, Lentinus similis
  REGION 1..248: note = peptide - Lentinus similis LPMO
HTLVWGVWVNGVDQGDGRNIYIRSPPNNNPVKNLTSPDMTCNVDNRVVPKSVPVNAGDTLTFEWYHNTRDDDIIASSHHGPIAVYIAPAASNGQGNVWVKLFEDAYNVTNSTWAVDRLITAHGQHSVVVPHVAPGDYLFRAEIIALHEADSLYSQNPIRGAQFYISCAQITINSSDDSTPLPAGVPFPGAYTDSTPGIQFNIYTTPATSYVAPPPSVWSGALGGSIAQVGDASLEENLYFQGHHHHHH

SEQ ID NO: 16 - DNA, 773 bp, N. crassa
  gene 1..773: note = Gene encoding Neurospora crasse LMPO
  source 1..773: mol_type = genomic DNA
atgaaggtcctcgcccctctcgtactcgcaagcgcagccagcgctcacaccattttctcctccctcgaggtcaacggcgtcaaccaaggcttgggagagggcgtccgcgtgcccacctacaacggtcccattgaggacgtcacctcggcctccatcgcctgcaacggctcgcccaacaccgtcgcctccacctccaaggtgatcaccgtgcaggcgggcaccaacgtgacggccatctggcgctacatgctcagcaccacgggcgactcgccggcggacgtcatggacagctcgcacaagggtcccaccatcgcctacctcaaaaaggttgacaacgccgccaccgccagcggtgtggggaatggctggttcaagatccagcaggacggcatggacagcagcggcgtctggggcaccgagcgcgttatcaacggcaagggccgccacagcatcaagatccccgagtgcatcgctccaggacagtacttactcagggctgagatgattgcgctgcacgcggcgagcaactatcctggtgcgcaattctacatggagtgtgcgcagcttaatgtcgttggtggtacgggtgctaagaccccttcgactgtcagctttcctggggcttactcggtatgtacaaccatttttacgcaggtgggatgattgctaacaaatgtttttcacagggctctgaccccggagtcaagattagcatctactggcctccggttacgtcttataccgtccctggtcccagtgtgtttacttgctaa

SEQ ID NO: 17 - protein, 238 aa, N. crassa
  REGION 1..238: note = peptide - N. crassa LPMO
MKVLAPLVLASAASAHTIFSSLEVNGVNQGLGEGVRVPTYNGPIEDVTSASIACNGSPNTVASTSKVITVQAGTNVTAIWRYMLSTTGDSPADVMDSSHKGPTIAYLKKVDNAATASGVGNGWFKIQQDGMDSSGVWGTERVINGKGRHSIKIPECIAPGQYLLRAEMIALHAASNYPGAQFYMECAQLNVVGGTGAKTPSTVSFPGAYSGSDPGVKISIYWPPVTSYTVPGPSVFTC

SEQ ID NO: 18 - DNA, 1016 bp, Heterobasidion irregulare
  gene 1..1016: note = Gene encoding Heterobasidion irregulare LPMO
  source 1..1016: mol_type = genomic DNA
atgttcctagtcccccttctggctgctctctctcttagtgcccccaaggtattcacgcatacttgccgtcacccggacatggactgatacctcaaaggttgccgcacatggtggtgtactagcttatagcctcgccggcacttggtacaatgggttcgtctcatcctcattgtcgactttatttcgcctgctcattctatttttttccagctttgtgccttataacactcctaccggtcaaagcaccatccagcgcgaatgggacacttacaatcccatcactgaccctaccgatgcctccatctcctgcaacatcaacggcgcttcactaggctccgcacaaaagtctgctactgtagcggcaggatcttccgtaactgcgtactggaagtgcgtgaaccgttgtcttacggtcaagcgggggtttatcgctgacactgacatctagtcaatggcctcacaccattggccccgtcatggtctacatggccaactgtggtggtgattgcacgaccgctaccacatcatctttggaatgggtatgcttcctcgtgaatcctccgattaagagcgctctcagttcatttctcgcaacagttcaagatcaaccaggttggtttggtctccggaacgctcacgagtggtacctggggtatgggccagctcgtcgcaaacaacaattcctggaccacttcaattccttcttcgcttgcagcgggcaactacatactccgccacgaactattggccattcacacctctaaccaaccccaattctacccggagtgcgcgcagctgattgtcaccggcggcgaaggagcgacccctccggctagttacctcgtcaagcttcctggtgcttattcgatgtatgtgcatcccaattggtatttgcgaatgtgtgttcaatttcgacaacttcgggcaggtctgaccctggtgtcaacattgatatctactcccatgagacggagacgaactacactattccgggacctgcggtctggcaaggatga

SEQ ID NO: 19 - protein, 244 aa, Heterobasidion irregulare
  REGION 1..244: note = peptide - Heterobasidion irregulare LPMO
MFLVPLLAALSLSAPKVAAHGGVLAYSLAGTWYNGFVPYNTPTGQSTIQREWDTYNPITDPTDASISCNINGASLGSAQKSATVAAGSSVTAYWNQWPHTIGPVMVYMANCGGDCTTATTSSLEWFKINQVGLVSGTLTSGTWGMGQLVANNNSWTTSIPSSLAAGNYILRHELLAIHTSNQPQFYPECAQLIVTGGEGATPPASYLVKLPGAYSMSDPGVNIDIYSHETETNYTIPGPAVWQG

SEQ ID NO: 20 - DNA, 1026 bp, Thermothelomyces thermophilus
  gene 1..1026: note = Gene encoding Thermothelomyces thermophilus LPMO
  source 1..1026: mol_type = genomic DNA
atgctgacaacaaccttcgccctcctgacggccgctctcggcgtcagcgcccattataccctccccagggtcgggaccggttccgactggcagcacgtgcggcgggctgacaactggcaaaacaacggcttcgtcggcgacgtcaactcggagcagatcaggtgcttccaggcgacccctgccggcgcccaagacgtctacactgttcaggcgggatcgaccgtgacctaccacgccaaccccagtatctaccaccccggccccatgcagttctacctggcccgcgttccggacggacaggacgtcaagtcgtggaccggcgagggtgccgtgtggttcaaggtgtacgaggagcagcctcaatttggcgcccagctgacctggcctagcaacggtgcgttgatcattttccttcttcttccttctttcttccgttgcatatgctaactgttctcttgcttgcaggcaagagctcgttcgaggttcctatccccagctgcattcgggcgggcaactacctcctccgcgctgagcacatcgccctgcacgttgcccaaagccagggcggcgcccagttctacatctcgtgcgcccagctccaggtcactggtggcggcagcaccgagccttctcagaaggtttccttcccgggtgcctacaagtccaccgaccccggcattcttatcaacatcaactaccccgtccctacctcgtaccagaatccgggtccggctgtcttccgttgctaagcgggcacaacaagggaattggaacggggcaatgggggcgggctggagccgcctgcgtccttgttttcttaccctcgccgttcgtacgaatcagggccaagcaaaacatcaaggaagggcgggggagggaagattccaacctaccaacttgggaagccgggggagaattctttgacgtggttgaccggctcagatcgaggagagcaacccaatctgtacatagacgcctatccttcacgatgaacatagtgccaatggagctcacgacagtcacacc

SEQ ID NO: 21 - protein, 225 aa, Thermothelomyces thermophilus
  REGION 1..225: note = peptide - Thermothelomyces thermophilus LPMO
MLTTTFALLTAALGVSAHYTLPRVGTGSDWQHVRRADNWQNNGFVGDVNSEQIRCFQATPAGAQDVYTVQAGSTVTYHANPSIYHPGPMQFYLARVPDGQDVKSWTGEGAVWFKVYEEQPQFGAQLTWPSNGKSSFEVPIPSCIRAGNYLLRAEHIALHVAQSQGGAQFYISCAQLQVTGGGSTEPSQKVSFPGAYKSTDPGILININYPVPTSYQNPGPAVFRC

SEQ ID NO: 22 - DNA, 934 bp, Podospora anserina
  gene 1..934: note = Gene encoding Podospora anserina LMPO9E
  source 1..934: mol_type = genomic DNA
atgaagggactcttgagcgttgccgccctttcgctggctgtcagcgaggtttcggcccactacatcttccagcagctctcgaccggcagcaccaagcacggcgtgttccagtacatccgtcaaaataccaactataatagccctgtgaccgacctcagctccaacgacctcaggtgtaacgagggtggtgccagcggtgccaatactcagaccgtgaccgtccgtgctggggactctttcaccttccacttggacactcctggtatgtcatcctaatcagccaaaccatttatacttagtctaacaccccatagtctaccaccagggtcctgtctccgtttacctcagcaaggccccaggctcagcttccagctacgacggcagcggcacctggttcaagatcaaggactggggcccgaccttccccggcggccagtggactctcgctggttcctacactgctcagcttcccagctgcatcaccgacggagagtaccttctccgcatccagtccctcggcatccacaacccataccccgccggtactccccagttttacatctcctgcgcccagatcaaggtcactggtggtggcagcgtgaacccatccggtgttgctatccccggtgccttcaaggccaccgaccccggctacaccgccaatatctacagcaacttcaactcctacaccgtccccggcccatccgtcttctcttgcggctccaacggcggtggcagctcccccgttgagccccagcctcagcccaccaccactctcgtcacctctacccgtgctcccgtcgccacccagcccgctggctgcgccgttgccaagtggggacagtgcggtggtaacgggtggactggctgcaccacttgcgctgctggctctacctgcaacacccagaacgcttactaccaccaatgcgtctaa

SEQ ID NO: 23 - protein, 293 aa, Podospora anserina
  REGION 1..293: note = peptide - Podospora anserina LPMO9E
MKGLLSVAALSLAVSEVSAHYIFQQLSTGSTKHGVFQYIRQNTNYNSPVTDLSSNDLRCNEGGASGANTQTVTVRAGDSFTFHLDTPVYHQGPVSVYLSKAPGSASSYDGSGTWFKIKDWGPTFPGGQWTLAGSYTAQLPSCITDGEYLLRIQSLGIHNPYPAGTPQFYISCAQIKVTGGGSVNPSGVAIPGAFKATDPGYTANIYSNFNSYTVPGPSVFSCGSNGGGSSPVEPQPQPTTTLVTSTRAPVATQPAGCAVAKWGQCGGNGWTGCTTCAAGSTCNTQNAYYHQCV

SEQ ID NO: 24 - DNA, 1113 bp, Podospora anserina
  gene 1..1113: note = Gene encoding Podospora anserina LPMO9H
  source 1..1113: mol_type = genomic DNA
atgaaggccttcactctcgtctccctggctgcgtccgtcagcgcccactccatcttccaaaaggtgtcggttaacggcgtcgaccaaggccagctcaagggcgtccgcgctccctacagcaacttccccatcgagaacgtcaaccaccccgactttgcctgcaacaccaacatccagctgcgggataacaccgtgatcaaggtcccagccggcgccagagtcggtgcctggtgggggcatgagattggtggtgccgccgggcccaacgacccggatcaccccatcgccgcctcccacaagggtcccatccaggtctacctcgccaaggtcaacaatgctgccaacgccggcacgtccggcctccagtggttcaaggtcgccgagcaaggcctcaacaacggcgtctgggctgtcgacaacatgatctccaacggcggctggcactacttcgacatgcccagctgcgtcgcccccggccactacctcatgcgcgtcgagctgctcgcccttcactccgccagcgtccgcggtgccgcccagttctacatggagtgcgcccagatcgagattactgggtctggcaccaacaccggctccaactttgtcagcttccccggtgcttacaccgcggaccaccccggcatcttggtctccatctatgacctccagggccgccctaccaacggcggccgtccttacaccatccccggccctgctcctctcacctgctccggtggctccaaccccaacccccagcctcagcccacttctgctgctcctaacccccagcctactggtggaaacggtggtggtgccggtgctcctctttatggccagtgcggtggtcaggggtataccggccctaccacctgtgctcagggtacttgcgtggcttccaaccagtggtacagtatgttgtcccccttatccttctttgcctcacacatgctgacgtgtctttcctccaggccagtgcctcccataaatgtctcaatgcgactcggaaaagggcacgagcatgagcgacgtcggattaataccaacaaacatcttattttcacctgtatatactttagcattttgccacctgacataccaggcggtagtacataa

SEQ ID NO: 25 - protein, 370 aa, Podospora anserina
  REGION 1..370: note = peptide - Podospora anserina LPMO9H
MKAFTLVSLAASVSAHSIFQKVSVNGVDQGQLKGVRAPYSNFPIENVNHPDFACNTNIQLRDNTVIKVPAGARVGAWWGHEIGGAAGPNDPDHPIAASHKGPIQVYLAKVNNAANAGTSGLQWFKVAEQGLNNGVWAVDNMISNGGWHYFDMPSCVAPGHYLMRVELLALHSASVRGAAQFYMECAQIEITGSGTNTGSNFVSFPGAYTADHPGILVSIYDLQGRPTNGGRPYTIPGPAPLTCSGGSNPNPQPQPTSAAPNPQPTGGNGGGAGAPLYGQCGGQGYTGPTTCAQGTCVASNQWYSMLSPLSFFASHMLTCLSSRPVPPINVSMRLGKGHEHERRRINTNKHLIFTCIYFSILPPDIPGGST

SEQ ID NO: 26 - DNA, 90 bp, homo sapiens
  source 1..90: mol_type = genomic DNA
  CDS 1..90: note = GLP-1 (7-36)
catgctgaagggacctttaccagtgatgtaagttcttatttggaaggccaagctgccaaggaattcattgcttggctggtgaaaggccga

SEQ ID NO: 27 - protein, 30 aa, homo sapiens
HAEGTFTSDVSSYLEGQAAKEFIAWLVKGR

SEQ ID NO: 28 - DNA, 93 bp, homo sapiens
  source 1..93: mol_type = genomic DNA
  CDS 1..93: note = GLP-1 (7-37)
catgctgaagggacctttaccagtgatgtaagttcttatttggaaggccaagctgccaaggaattcattgcttggctggtgaaaggccgaggc

SEQ ID NO: 29 - protein, 31 aa, homo sapiens
HAEGTFTSDVSSYLEGQAAKEFIAWLVKGRG

SEQ ID NO: 30 - DNA, 57 bp, Lentinus similis
  sig_peptide 1..57: note = Lentinus similis LPMO native signal peptide
  source 1..57: mol_type = genomic DNA
atgaagtactccatcctcgggctcacggcactttccttcgtcgcgtcagccgcggct

SEQ ID NO: 31 - protein, 18 aa, L. similis
  SIGNAL 1..18: note = Lentinus similis LPMO native signal peptide
MKYSILGLTALSFVASAA

SEQ ID NO: 32 - DNA, 66 bp, S. cerevisiae
  sig_peptide 1..66: note = OST1 signal peptide
  source 1..66: mol_type = genomic DNA
atgaggcaggtttggttctcttggattgtgggattgttcctatgttttttcaacgtgtcttctgct

SEQ ID NO: 33 - protein, 22 aa, S. cerevisiae
  SIGNAL 1..22: note = OST1 signal peptide
MRQVWFSWIVGLFLCFFNVSSA

SEQ ID NO: 34 - DNA, 60 bp, A. nidulans
  sig_peptide 1..60: note = A. nidulans amySP signal peptide
  source 1..60: mol_type = genomic DNA
atggtcgcttggtggtctttgtttctgtacggtcttcaggtcgctgcacctgctttggct

SEQ ID NO: 35 - protein, 20 aa, A. nidulans
  SIGNAL 1..20: note = A. nidulans amySP signal peptide
MVAWWSLFLYGLQVAAPALA

SEQ ID NO: 36 - DNA, 132 bp, synthetic construct
  source 1..132: mol_type = other DNA
  CDS 1..132: note = Lixisenatide
catggtgaaggtacttttacttctgatttgtctaaacaaatggaagaagaagctgttagattgtttattgaatggttgaaaaatggtggtccatcttctggtgctccaccatctaaaaaaaaaaaaaaaaaa

SEQ ID NO: 37 - protein, 44 aa, synthetic construct
HGEGTFTSDLSKQMEEEAVRLFIEWLKNGGPSSGAPPSKKKKKK

SEQ ID NO: 38 - DNA, 117 bp, synthetic construct
  source 1..117: mol_type = other DNA
  CDS 1..117: note = Exanatide
catggtgaaggtacttttacttctgatttgtctaaacaaatggaagaagaagctgttagattgtttattgaatggttgaaaaatggtggtccatcttctggtgctccaccaccatct

SEQ ID NO: 39 - protein, 39 aa, synthetic construct
HGEGTFTSDLSKQMEEEAVRLFIEWLKNGGPSSGAPPPS

SEQ ID NO: 40 - DNA, 93 bp, synthetic construct
  source 1..93: mol_type = other DNA
  CDS 1..93: note = Liraglutide
catgctgaaggtacttttacttctgatgtttcttcttatttggaaggtcaagctgctaaagaatttattgcttggttggttagaggtagaggt

SEQ ID NO: 41 - protein, 31 aa, synthetic construct
HAEGTFTSDVSSYLEGQAAKEFIAWLVRGRG

SEQ ID NO: 42 - DNA, 1935 bp, synthetic construct
  source 1..1935: mol_type = other DNA
  CDS 1..1935: note = Albiglutide
catggtgaaggtacttttacttctgatgtttcttcttatttggaaggtcaagctgctaaagaatttattgcttggttggttaaaggtagacatggtgaaggtacttttacttctgatgtttcttcttatttggaaggtcaagctgctaaagaatttattgcttggttggttaaaggtagagatgctcataaatctgaagttgctcatagatttaaagatttgggtgaagaaaattttaaagctttggttttgattgcttttgctcaatatttgcaacaatgtccatttgaagatcatgttaaattggttaatgaagttactgaatttgctaaaacttgtgttgctgatgaatctgctgaaaattgtgataaatctttgcatactttgtttggtgataaattgtgtactgttgctactttgagagaaacttatggtgaaatggctgattgttgtgctaaacaagaaccagaaagaaatgaatgttttttgcaacataaagatgataatccaaatttgccaagattggttagaccagaagttgatgttatgtgtactgcttttcatgataatgaagaaacttttttgaaaaaatatttgtatgaaattgctagaagacatccatatttttatgctccagaattgttgttttttgctaaaagatataaagctgcttttactgaatgttgtcaagctgctgataaagctgcttgtttgttgccaaaattggatgaattgagagatgaaggtaaagcttcttctgctaaacaaagattgaaatgtgcttctttgcaaaaatttggtgaaagagcttttaaagcttgggctgttgctagattgtctcaaagatttccaaaagctgaatttgctgaagtttctaaattggttactgatttgactaaagttcatactgaatgttgtcatggtgatttgttggaatgtgctgatgatagagctgatttggctaaatatatttgtgaaaatcaagattctatttcttctaaattgaaagaatgttgtgaaaaaccattgttggaaaaatctcattgtattgctgaagttgaaaatgatgaaatgccagctgatttgccatctttggctgctgattttgttgaatctaaagatgtttgtaaaaattatgctgaagctaaagatgtttttttgggtatgtttttgtatgaatatgctagaagacatccagattattctgttgttttgttgttgagattggctaaaacttatgaaactactttggaaaaatgttgtgctgctgctgatccacatgaatgttatgctaaagtttttgatgaatttaaaccattggttgaagaaccacaaaatttgattaaacaaaattgtgaattgtttgaacaattgggtgaatataaatttcaaaatgctttgttggttagatatactaaaaaagttccacaagtttctactccaactttggttgaagtttctagaaatttgggtaaagttggttctaaatgttgtaaacatccagaagctaaaagaatgccatgtgctgaagattatttgtctgttgttttgaatcaattgtgtgttttgcatgaaaaaactccagtttctgatagagttactaaatgttgtactgaatctttggttaatagaagaccatgtttttctgctttggaagttgatgaaacttatgttccaaaagaatttaatgctgaaacttttacttttcatgctgatatttgtactttgtctgaaaaagaaagacaaattaaaaaacaaactgctttggttgaattggttaaacataaaccaaaagctactaaagaacaattgaaagctgttatggatgattttgctgcttttgttgaaaaatgttgtaaagctgatgataaagaaacttgttttgctgaagaaggtaaaaaattggttgctgcttctcaagctgctttgggtttg

SEQ ID NO: 43 - protein, 645 aa, synthetic construct
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 44 - DNA, 825 bp, synthetic construct
  source 1..825: mol_type = other DNA
  CDS 1..825: note = Dulaglutide
catggtgaaggtacttttacttctgatgtttcttcttatttggaagaacaagctgctaaagaatttattgcttggttggttaaaggtggtggtggtggtggtggttctggtggtggtggttctggtggtggtggttctgctgaatctaaatatggtccaccatgtccaccatgtccagctccagaagctgctggtggtccatctgtttttttgtttccaccaaaaccaaaagatactttgatgatttctagaactccagaagttacttgtgttgttgttgatgtttctcaagaagatccagaagttcaatttaattggtatgttgatggtgttgaagttcataatgctaaaactaaaccaagagaagaacaatttaattctacttatagagttgtttctgttttgactgttttgcatcaagattggttgaatggtaaagaatataaatgtaaagtttctaataaaggtttgccatcttctattgaaaaaactatttctaaagctaaaggtcaaccaagagaaccacaagtttatactttgccaccatctcaagaagaaatgactaaaaatcaagtttctttgacttgtttggttaaaggtttttatccatctgatattgctgttgaatgggaatctaatggtcaaccagaaaataattataaaactactccaccagttttggattctgatggttctttttttttgtattctagattgactgttgataaatctagatggcaagaaggtaatgttttttcttgttctgttatgcatgaagctttgcataatcattatactcaaaaatctttgtctttgtctttgggt

SEQ ID NO: 45 - protein, 275 aa, synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 46 - protein, 31 aa, synthetic construct
  REGION 1..31: note = peptide - Semaglutide; X is Aib (2-Aminoisobutyric acid (Aib), or ?-methylalanine).
HXEGTFTSDVSSYLEGQAAKEFIAWLVRGRG

SEQ ID NO: 47 - DNA, 1677 bp, synthetic construct
  source 1..1677: mol_type = other DNA
  CDS 1..1677: note = AN4663 codon optimized for expression in Pichia pastoris (K. phaffii)
atggcgccttttcgttcaatttatgagaaagatgccaccaaaaagcttgttgtaggagcagcgttgcttgtgctggctgcgttttatagttatgtatttctgctgactctggcgcctgtatatggttctactccttcccatatctttcacggctatggagtcggtatcgcgggtgtagctggctggttttcgaaggacattgtggatcgcgtaagcggtcgtaaagcaatctatgcaattccagttttggcgttctttttaccagtcgttcaatactttgtcagtcagcagtcatctgcacttggcaatcctgcggggccaatcttcacagaggtattggctctgtaccctctggtcttgttatcagttgcttgtgcggggaagctggtccaggccggtcttaacttgcaacgccatggagacttggtagctgaacacattcctttacttgggtcgtacgttatttattctgcaggggagcatcttatcaaggccttcttatctcgcttcatcggctccaccgtacttttgtcacgcgctggcttacagattcttatcgcaattttctacgctgccgccgttccttccaaggcgttacttctggccattccagcgttcttattctcagtcacatcgaacacccaccttcctttgggccacacaacgaccgcacttaataatatcatcgctgatgacggcttcgcacttgttgcacgccaagacagcactaccgggtacatttcagtcttggataaccttgaggatgggtttcgcgtaatgcgctgtgaccacagtttactgggaggtcaatggatcaaaaaacgtcccaattatactcctccagccgtaaaggaccccatctatgcagtattcacaatgttggaggcagtgcgcttggtcgaaacggctcacggtatcccccgtgctgatgcgggctccaacgcgcttgtaatcggcttggggatcggcactacccctggcgctttgattagtcacggaattgacaccacgattgtcgaaattgaccctgttgtccacaaatatgcccttcaatattttgaccttcctgaaaaccacacacctatcattgaggacgcacgcgcttttgtacagcgttcgcgtaatgctccacaacccaagcagtacgattacattgtgcatgacgtgttcactggcggagctgagccggtagagttgttcacctatgagttcatctcgggcctgcatgcgcttttgaaagacgatggagttatcgccattaactacgcaggggatatttccttatattcaacagctctgagcatccgcacaatcaaaagcatttttcccacctgtcgcttgttccgcgaggctgccgccccggagatcggaccggattttacgaatatggtcattttctgcacgaaatcgcgtggtgcaccgattacattccgcgatcccgtaccggaagatttcttgggaagccgctttcgttctcgttaccttgttccaaaacatgaggtagatgccgcgcaattcgacaacgtcgggttggaagacggtcctcagggacatggccgccgcgtgctggttgacaaagaggtcggtcgtttacacaaatatcaggaccgttccgcactggagcattggggaattatgcgtaccgtcttgccagatcgcgtctgggagggctggtga

SEQ ID NO: 48 - protein, 558 aa, synthetic construct; identical to SEQ ID NO: 2

SEQ ID NO: 49 - DNA, 6608 bp, synthetic construct
  misc_feature 5490..6506: note = cytosolic AN4663 (codon optimized)
  source 1..6608: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagggcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattatttgtcctactcaggagagcgttcaccgacaaacaacagataaaacgaaaggcccagtctttcgactgagcctttcgttttatttggcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcagatcttcgtgaaaacactgaccggcaaaaccattaccctggaggtggagcctggcagcgcccatcatcatcaccatcaccatcaccatcatgctggtagcagcgatacgatcgaaaacgtcaaagccaaaattcaggacaaagagggcattccgcctgatcaacaacgtctgatcttcgccggaaaacagctggaggatggccgtacactgagtgactataacattcaagagaaatcgacactggaactggttctggagctgcagaatgaaacccagtcagcaacttctgacgcttctggcggtgaaaacctgtacttccaacatacaacgaccgcacttaataatatcatcgctgatgacggcttcgcacttgttgcacgccaagacagcactaccgggtacatttcagtcttggataaccttgaggatgggtttcgcgtaatgcgctgtgaccacagtttactgggaggtcaatggatcaaaaaacgtcccaattatactcctccagccgtaaaggaccccatctatgcagtattcacaatgttggaggcagtgcgcttggtcgaaacggctcacggtatcccccgtgctgatgcgggctccaacgcgcttgtaatcggcttggggatcggcactacccctggcgctttgattagtcacggaattgacaccacgattgtcgaaattgaccctgttgtccacaaatatgcccttcaatattttgaccttcctgaaaaccacacacctatcattgaggacgcacgcgcttttgtacagcgttcgcgtaatgctccacaacccaagcagtacgattacattgtgcatgacgtgttcactggcggagctgagccggtagagttgttcacctatgagttcatctcgggcctgcatgcgcttttgaaagacgatggagttatcgccattaactacgcaggggatatttccttatattcaacagctctgagcatccgcacaatcaaaagcatttttcccacctgtcgcttgttccgcgaggctgccgccccggagatcggaccggattttacgaatatggtcattttctgcacgaaatcgcgtggtgcaccgattacattccgcgatcccgtaccggaagatttcttgggaagccgctttcgttctcgttaccttgttccaaaacatgaggtagatgccgcgcaattcgacaacgtcgggttggaagacggtcctcagggacatggccgccgcgtgctggttgacaaagaggtcggtcgtttacacaaatatcaggaccgttccgcactggagcattggggaattatgcgtaccgtcttgccagatcgcgtctgggagggctggtaagatccggctccaccgctgagcaataactagcataaccccttggggcctctaaacgggtcttgaggggttttttgctgaaaggaggaactatatccggat

SEQ ID NO: 50 - DNA, 9780 bp, synthetic construct
  misc_feature 1006..1710: note = LsAA9A
  misc_feature 2484..5018: note = Complementary strand: Pichia pastoris HIS4 "multifunctional enzyme, required for histidine biosynthesis"
  source 1..9780: mol_type = other DNA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgaagtactccatcctcgggctcacggcactttccttcgtcgcgtcagccgcggctcataccctcgtctggggcgtatgggtcaacggcgttgatcaaggagatgggaggaacatctacatcaggtctcccccgaacaataaccccgtcaagaacctgacttcgccggatatgacgtgcaatgtcgacaatcgcgttgtgcccaagagcgtgccagtcaacgctggtgatacgcttactttcgaatggtaccacaacacacgagacgacgatatcatcgcatcttctcatcacggacccatcgccgtatacatcgcacctgccgcttcgaacggtcaagggaacgtttgggtgaagctgttcgaggacgcctacaacgtcaccaatagcacttgggctgtcgatcgtctcatcactgctcatggacaacactccgttgtggtcccccatgttgcacctggagactacctatttagagctgagatcattgcgctacatgaggcagattctctgtactcccagaaccctatccgtggcgcgcaattttatatctcgtgcgctcaaatcaccatcaactcttccgatgactcgacacctctccccgctggtgttcccttccccggtgcatacaccgactccacacccggtattcaattcaatatctacactacccccgcgacaagctacgttgctccccctcccagcgtatggtctggagcgttgggtgggtcgattgctcaggtcggagatgcttctctcgaggaaaacctctacttccagggccatcatcaccatcaccactaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcaaccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 51 - DNA, 9783 bp, synthetic construct
  misc_feature 1009..1713: note = LsAA9A
  misc_feature 2487..5021: note = Complementary strand: Pichia pastoris HIS4
  source 1..9783: mol_type = other DNA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatggtcgcttggtggtctttgtttctgtacggtcttcaggtcgctgcacctgctttggctcataccctcgtctggggcgtatgggtcaacggcgttgatcaaggagatgggaggaacatctacatcaggtctcccccgaacaataaccccgtcaagaacctgacttcgccggatatgacgtgcaatgtcgacaatcgcgttgtgcccaagagcgtgccagtcaacgctggtgatacgcttactttcgaatggtaccacaacacacgagacgacgatatcatcgcatcttctcatcacggacccatcgccgtatacatcgcacctgccgcttcgaacggtcaagggaacgtttgggtgaagctgttcgaggacgcctacaacgtcaccaatagcacttgggctgtcgatcgtctcatcactgctcatggacaacactccgttgtggtcccccatgttgcacctggagactacctatttagagctgagatcattgcgctacatgaggcagattctctgtactcccagaaccctatccgtggcgcgcaattttatatctcgtgcgctcaaatcaccatcaactcttccgatgactcgacacctctccccgctggtgttcccttccccggtgcatacaccgactccacacccggtattcaattcaatatctacactacccccgcgacaagctacgttgctccccctcccagcgtatggtctggagcgttgggtgggtcgattgctcaggtcggagatgcttctctcgaggaaaacctctacttccagggccatcatcaccatcaccactaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcaaccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 52 - DNA, 9505 bp, synthetic construct
  misc_feature 1..9505: note = plasmid pLyGo-Kp-2
  source 1..9505: mol_type = other DNA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatggtcgcttggtggtctttgtttctgtacggtcttcaggtcgctgcacctgctttggctcatcgaagagcttaattatattccccagaacatcaggttaatggcgtttttgatgtcattttcgcggtggctgagatcagccacttcttccccgataacggagaccggcacactggccatatcggtggtcatcatgcgccagcttttatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatagcatacctctttttgacatacttcgggtatacatatcagtatatattcttataccgcaaaaatcagcgcgcaaatacgcatactgttatctggcttttagtagcgaaaaaaccccgccgaagcggggttttttgcggctcttcgtaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcaaccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 53 - DNA, 1827 bp, human
  source 1..1827: mol_type = genomic DNA
  CDS 1..1827: note = HSA (human serum albumin)
atgaagtgggtaacgtttatctctcttttatttcttttctcatccgcatattctaggggcgtttttaggagggacgcccacaagagtgaggttgcacacagatttaaggacttgggtgaagagaatttcaaagcattggtccttatagctttcgcacagtatttgcagcaatgcccatttgaagatcatgtaaagctggtgaacgaggtcactgagttcgccaagacctgcgtcgcagacgaatccgccgagaattgtgataaatctcttcacacgctattcggtgataagctatgtaccgtggcaacgttaagggaaacttacggcgagatggcagactgctgtgcaaaacaagagcccgagaggaacgaatgttttctacaacataaagacgacaatcctaatttgcctagactagtaagacccgaggtcgatgttatgtgtaccgccttccatgacaatgaggagacgttcctaaaaaagtatctatacgagattgcaagaaggcatccatatttctacgcccccgagctactgttttttgcaaaaaggtacaaagcagccttcactgaatgctgtcaagcagctgacaaagccgcttgtctattaccaaagctggatgagctaagagatgaaggtaaagcctcctctgctaagcaacgtttaaagtgcgcatccctgcaaaagtttggcgaacgtgcattcaaggcctgggccgtcgccaggctttcacaaagattcccaaaagccgaattcgctgaagtgtctaaactggtcacggacttgacaaaagttcacacagaatgttgtcacggcgacctacttgagtgcgcagatgacagggccgacctagctaagtatatttgtgagaatcaggattcaatctcctccaaacttaaggagtgttgtgagaaacctctgttagagaagtctcactgtatcgctgaggtggagaacgacgaaatgccagccgacctgccttctctggctgctgattttgttgagtccaaggatgtttgcaaaaactacgctgaggcaaaagacgtgttcctaggcatgttcttgtacgaatatgctagaaggcaccccgactattctgtcgtactgctgttgcgtcttgccaagacgtacgaaactacactagagaaatgctgtgccgcagcagatcctcatgagtgctacgcaaaagtgttcgatgaattcaaacccctagtggaagaaccccaaaacttgataaaacagaactgcgaactatttgaacaactgggcgaatacaagttccaaaatgccttgttagtacgttatactaaaaaagttccacaagtatccacgcctaccttagtcgaggtaagtaggaatttgggtaaagttggctcaaagtgctgcaagcacccagaggctaaacgtatgccatgcgcagaagactatttaagtgtggttttgaaccagctttgcgttctgcacgaaaagactccagtttctgatagggtcacaaaatgctgtaccgagagtctagttaaccgtagaccttgcttttccgccttggaggttgacgaaacgtacgttcctaaggagttcaatgcagaaacttttacattccacgcagacatttgcacactttctgaaaaagagcgtcagataaaaaaacaaacagcccttgtagagttggtgaagcacaagcctaaggctactaaggagcaacttaaagctgtaatggatgacttcgccgccttcgttgaaaagtgttgcaaagccgacgataaggaaacatgtttcgcagaggagggaaagaagttagtcgccgctagtcaggccgctcttggccta

SEQ ID NO: 54 - protein, 609 aa, human [sequence omitted]

SEQ ID NO: 55 - DNA, 1980 bp, synthetic construct
  source 1..1980: mol_type = other DNA
  CDS 1..1980: note = AmySP-GLP1-HSA
atggtcgcttggtggtctttgtttctgtacggtcttcaggtcgctgcacctgctttggctcatgctgaagggacctttaccagtgatgtaagttcttatttggaaggccaagctgccaaggaattcattgcttggctggtgaaaggccgaatgaagtgggtaacgtttatctctcttttatttcttttctcatccgcatattctaggggcgtttttaggagggacgcccacaagagtgaggttgcacacagatttaaggacttgggtgaagagaatttcaaagcattggtccttatagctttcgcacagtatttgcagcaatgcccatttgaagatcatgtaaagctggtgaacgaggtcactgagttcgccaagacctgcgtcgcagacgaatccgccgagaattgtgataaatctcttcacacgctattcggtgataagctatgtaccgtggcaacgttaagggaaacttacggcgagatggcagactgctgtgcaaaacaagagcccgagaggaacgaatgttttctacaacataaagacgacaatcctaatttgcctagactagtaagacccgaggtcgatgttatgtgtaccgccttccatgacaatgaggagacgttcctaaaaaagtatctatacgagattgcaagaaggcatccatatttctacgcccccgagctactgttttttgcaaaaaggtacaaagcagccttcactgaatgctgtcaagcagctgacaaagccgcttgtctattaccaaagctggatgagctaagagatgaaggtaaagcctcctctgctaagcaacgtttaaagtgcgcatccctgcaaaagtttggcgaacgtgcattcaaggcctgggccgtcgccaggctttcacaaagattcccaaaagccgaattcgctgaagtgtctaaactggtcacggacttgacaaaagttcacacagaatgttgtcacggcgacctacttgagtgcgcagatgacagggccgacctagctaagtatatttgtgagaatcaggattcaatctcctccaaacttaaggagtgttgtgagaaacctctgttagagaagtctcactgtatcgctgaggtggagaacgacgaaatgccagccgacctgccttctctggctgctgattttgttgagtccaaggatgtttgcaaaaactacgctgaggcaaaagacgtgttcctaggcatgttcttgtacgaatatgctagaaggcaccccgactattctgtcgtactgctgttgcgtcttgccaagacgtacgaaactacactagagaaatgctgtgccgcagcagatcctcatgagtgctacgcaaaagtgttcgatgaattcaaacccctagtggaagaaccccaaaacttgataaaacagaactgcgaactatttgaacaactgggcgaatacaagttccaaaatgccttgttagtacgttatactaaaaaagttccacaagtatccacgcctaccttagtcgaggtaagtaggaatttgggtaaagttggctcaaagtgctgcaagcacccagaggctaaacgtatgccatgcgcagaagactatttaagtgtggttttgaaccagctttgcgttctgcacgaaaagactccagtttctgatagggtcacaaaatgctgtaccgagagtctagttaaccgtagaccttgcttttccgccttggaggttgacgaaacgtacgttcctaaggagttcaatgcagaaacttttacattccacgcagacatttgcacactttctgaaaaagagcgtcagataaaaaaacaaacagcccttgtagagttggtgaagcacaagcctaaggctactaaggagcaacttaaagctgtaatggatgacttcgccgccttcgttgaaaagtgttgcaaagccgacgataaggaaacatgtttcgcagaggagggaaagaagttagtcgccgctagtcaggccgctcttggcctataa

SEQ ID NO: 56 - protein, 659 aa, synthetic construct
MVAWWSLFLYGLQVAAPALAHAEGTFTSDVSSYLEGQAAKEFIAWLVKGRMKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 57 - protein, 10 aa, synthetic construct
  PEPTIDE 1..10: note = Sequence of the n-terminal peptide sequence monitored by the mass spectrometer
HTVIVYPGYR

SEQ ID NO: 58 - protein, 10 aa, synthetic construct
  PEPTIDE 1..10: note = Methylated HTVIVYPGYR sequence of the n-terminal peptide sequence monitored by the mass spectrometer
  MOD_RES 1: note = Methylation
HTVIVYPGYR

SEQ ID NO: 59 - protein, 18 aa, synthetic construct
  SIGNAL 1..10: note = Native Signal Peptide
  PEPTIDE 11..18: note = N-terminus of LsAA9A
ALSFVASAAAHTLVWGVW

SEQ ID NO: 60 - protein, 18 aa, synthetic construct
  SIGNAL 1..10: note = Amy Signal peptide
  PEPTIDE 11..18: note = N-terminus of LsAA9A
GLQVAAPALAHTLVWGVW

SEQ ID NO: 61 - protein, 10 aa, Aspergillus nidulans
ALVIGLGIGT

SEQ ID NO: 62 - protein, 10 aa, Homo sapiens
LLVVGLGGGS

SEQ ID NO: 63 - protein, 10 aa, Homo sapiens
ILSIGGGAGE

SEQ ID NO: 64 - protein, 10 aa, Trypanosoma cruzi
VLIIGGGDGG

SEQ ID NO: 65 - protein, 10 aa, Bacillus subtilis
VLVVGGGDGG

SEQ ID NO: 66 - protein, 5 aa, Aspergillus nidulans
VFTGG

(19) United States
(12) Patent Application Publication
(10) Pub. No.: US 2025/0236850 A1
(43) Pub. Date: Jul. 24, 2025

(54) HISTIDINE METHYLTRANSFERASE FOR INCREASED PEPTIDE AND PROTEIN STABILITY

(71) Applicants: DANMARKS TEKNISKE UNIVERSITET, Kongens Lyngby (DK); KØBENHAVNS UNIVERSITET, København K (DK)

(72) Inventors: Jonas Laurberg Simonsen, Kongens Lyngby (DK); Cristina Hemández Rollán, Kongens Lyngby (DK); Jakob Blæsbjerg Hoof, Kongens Lyngby (DK); Morten Helge Hauberg Nørholm, Kongens Lyngby (DK); Tanveer Singh Batth, København K (DK); Jesper Velgaard Olsen, København K (DK); Katja Salomon Johansen, København K (DK); Søren Brander, København K (DK)

(21) Appl. No.: 18/853,623

(22) PCT Filed: Apr. 4, 2023
(86) PCT No.: PCT/EP2023/058862

§ 371 Date: Oct. 2, 2024

(30) Foreign Application Priority Data
Apr. 8, 2022 (EP) 22167491.4

(51) Int. Cl. C12N 9/02; C12N 15/81

(52) U.S. Cl. CPC C12N 9/0083; C12N 15/815

(57) ABSTRACT

The present invention relates to an enzyme exhibiting histidine N-methyltransferase activity, a microbial cell comprising a gene encoding such enzyme, and use of this enzyme for producing a target peptide or protein having a methylated N-terminal histidine residue.

### FIELD OF THE INVENTION

The present invention relates to an enzyme exhibiting histidine N-methyltransferase activity, a microbial cell comprising a gene encoding such enzyme, and use of this enzyme for producing a target peptide or protein having a methylated N-terminal histidine residue.

### BACKGROUND OF THE INVENTION

Protein posttranslational modifications (PTMs) such as site-specific phosphorylation and methylation are important regulators of protein activity, stability, subcellular localization, and interactions. Protein methylation is a widely occurring PTM used to regulate DNA transcription via modification of histones at lysine and arginine residues in eukaryotic and prokaryotic organisms, constituting the primary component of the so-called “histone code”.

One such PTM is the targeted histidine methylation of highly abundant actin proteins detected in mammalian cells; where its primary function involves the regulation of actin filament formation. Targeted methylation of the imidazole ring of the N-terminal histidine residue of a protein is a methylation event found in living cells, that is prevalent in filamentous fungi but not found in many other micro-organisms such as yeast and bacteria.

Among the few reported N-terminal histidine methylation events, none are as important as those found in fungal lytic polysaccharide monooxygenases (LPMOs). LPMOs are a class of oxidative enzymes having broad substrate specificities towards complex polysaccharides including recalcitrant lignocellulose that are widely distributed in nature. LPMO activity plays an important role in facilitating the conversion of plant biomass as a renewable material resource for biofuel production. A common feature of all LPMOs is their N-terminal histidine, which is crucial for their copper binding and catalytic activity. However, a specific characteristic of LPMOs found in filamentous fungi is the targeted methylation of their N-terminal histidine residue, which is most commonly detected at one of the nitrogen atoms of the imidazole ring, N3 (T-methylation of Nε2) or N1 (or n), preferably N3. This N-terminal histidine methylation event, characteristic of filamentous fungal LPMOs, is both found to enhance their stability and their performance in an applied setting.

Members of the incretin hormone family (e.g. glucagon; GLP1) are proteins whose in vivo half-life is limited due to their rapid degradation by the DPP-IV enzyme. DPP-IV requires an intact α-amino-group of the N-terminal histidine of GLP-1 in order to degrade GLP-1. GLP-1 analogues having enhanced stability include those having a methylated N-terminal histidine (i.e. His7).

Genetically-modified cell factories are used to produce a wide diversity of commercially-important proteins; including biological pharmaceuticals and a diversity of enzymes. However, the choice of host as cell factory is limited when the host cells lack the PTM machinery required to produce proteins whose functional properties depend on PTMs. In respect to N-terminal histidine methylation, there are no known fungal S-adenosylmethionine-dependent methyltransferase enzymes capable of catalyzing this specific PTM, nor is this activity found in all potential cell factories (for example members of the Saccharomycetaceae family).

### SUMMARY OF THE INVENTION

In a first aspect, the present invention provides a genetically-modified cell for production of a target peptide or polypeptide having a modified N-terminal histidine residue, wherein the cell comprises:
a. a first gene comprising a first nucleic acid sequence encoding a first polypeptide exhibiting S-adenosylmethionine-dependent N-terminal histidine methyltransferase (NHMT) activity,
   wherein said first gene is genetically engineered,
   wherein said first polypeptide (i) is of fungal origin, (ii) is not native to said cell, (iii) comprises an N-terminal 7 transmembrane spanning domain, and (iv) comprises a soluble C-terminal NHMT catalytic domain, said catalytic domain comprising a SAM binding domain having a glutamate residue, and
b. a second gene comprising a second nucleic acid sequence encoding a target peptide or polypeptide, or a precursor thereof,
   wherein said target peptide or polypeptide comprises a N-terminal histidine residue; and
   wherein said first polypeptide facilitates modification of the N-terminal histidine residue of the target peptide or polypeptide, said modification comprising transfer of a methyl, dimethyl, ethyl or propyl group to the N-terminal histidine residue of the target peptide or polypeptide.

In a second aspect, the present invention provides an isolated polypeptide exhibiting histidine N-methyltransferase enzyme activity, wherein said polypeptide comprises an amino acid sequence having at least 60% sequence identity to amino acid residues 220 to 558 of SEQ ID NO.: 2.

In a third aspect, the present invention provides a method for production of a target peptide or polypeptide having a modified N-terminal histidine residue, comprising the steps of:
a. providing a genetically-modified cell according to the invention, or a cell population derived therefrom;
b. culturing the cell or cell population under conditions allowing expression of the first gene comprising a first nucleic acid sequence encoding a first polypeptide exhibiting N-terminal histidine methyltransferase activity and the second gene comprising a second nucleic acid sequence encoding a target peptide or polypeptide, or a precursor thereof,
c. recovering the target peptide or polypeptide having a modified N-terminal histidine residue.

In a fourth aspect, the present invention provides the use of the genetically-modified cell of the invention or the isolated polypeptide of the invention, for production of a target peptide or polypeptide having a modified N-terminal histidine residue.

### DESCRIPTION OF THE INVENTION

### Brief Description of the Figures

FIG. 1: (A) Schematic representation of the CRISPR-Cas9 knockout library generation. CRISPR-Cas9 plasmids expressing specific sgRNA, each targeting one of the methyltransferases candidate genes, were individually transformed into A. nidulans NID2531 strain. Each knockout strain was first cultured; the whole proteome was then extracted and subjected to a targeted proteomics assay (PMR) to detect N-terminal methylation of a synthetic peptide. (B) Knockout candidates were identified as genes encoding an N-terminal histidine methyltransferase based on the results of the PRM assay illustrated in the last step of FIG. 1A.

FIG. 2: (A) Data generated by the targeted proteomic assay (PMR; see FIG. 1) using mass spectrometry. Top graphs: an N-terminal histidine methylated peptide detected for the PRM assay of the reference A. nidulans strain (NID2531) (top-left graph), while a non-methylated peptide was not detected (top right graph). Middle graphs: the knockout strain candidate NID2713 (AN4663) failed to methylate the N-terminal histidine (middle-left graph), and the un-methylated peptide was instead identified (middle-right graph). Lower graphs: a strain expressing a mutant AN4663 with an E340A substitution in the SAM catalytic domain failed to methylate the N-terminal histidine (lower-left graph), and the un-methylated peptide was found instead (lower-right graph). (B) Graph showing the relative quantification of the N-terminal methylation histidine peptide (H-met-TVIVPGYR) detected by PMR assays of the reference A. nidulans strain (NID2531), the knockout AN4663 candidate (NID2713), the AN4663 candidate with E340A mutation (NID2787), and the AN4663 candidate carrying an mRFP tag at the N-terminus (NID2789) or at the C-terminus (NID2794). (C) Relative quantification of the un-methylation (HTVIVPGYR) detected by the PMR assays for the same strains as in FIG. 2B. (D) Alignment of representative sequences, focused on the spermidine synthase motif. NHMT=A. nidulans AN4663; METTL13=Human eEEF1 n-terminal methyl transferase; Histamine MT=Human histamine n-methyl transferase; SpeE (T. cruzi)=Trypanosoma cruzi spermidine synthase; and SpeE (B. sub)=Bacillus subtilis spermidine synthase. The residues corresponding to I322 are marked with a black box. The lack of an acidic group in this position shows that NHMT is not a spermidine synthase

FIG. 3: (A) Illustration of the predicted transmembrane and catalytic domain of AN4663 candidate NHMT. (B) AlphaFold2 model of AN4663 predicts four structural elements: 7TM, four stranded anti-parallel beta-sheet, Rossmann-like domain, and a helical extension. The model shows the N-terminal 7 trans-membrane spanning domain (amino acids: 1-230), a ß-sheet domain (amino acids: 233-288), the SAM binding domain (amino acids: 289-502) and a C-terminal tail (amino acids: 503-558). The three domains (amino acids: 233-558) in combination constitute the soluble catalytic domain.

FIG. 4: Schematic overview of LPMO LsAA9A and NHMT AN4663 co-expression in K. phaffii. A DNA construct encoding LsAA9A was inserted into the K. phaffii genome and expressed under control of the strong methanol-inducible promoter pAOX. NHMT was produced from an episomal plasmid.

FIG. 5: Schematic overview of synthesis of LPMO LsAA9A having either a native signal peptide (A) or an Amy signal peptide (B), showing signal peptide cleavage giving rise to an N-terminal histidine residue.

FIG. 6: LC-MS/MS analysis of LsAA9A secreted by K. phaffii expressing an LsAA9A gene with native signal peptide (A) or Amy signal peptide (B), showing the N-terminal sequence of expressed LsAA9A. The graph shows the intensity of the N-terminal peptide as number of hits identified from the LC-MS/MS analysis. Protein samples were digested with Trypsin and LysC to generate internal peptides of different lengths.

FIG. 7: SDS-PAGE (bottom) of the secreted LsAA9A into the supernatant from the co-expression of NHMT AN4663 and LsAA9A in K. phaffii; and LPMO activity (top graph) measured by the AZCL-HEC assay. E.V.=empty vector (negative control); NHMT=K. phaffii expressing NHMT; LsAA9A=K. phaffii expressing LPMO; LsAA9A+NHMT=K. phaffii co-expressing NHMT and LPMO. (A) LsAA9A gene with native signal peptide. (B) LsAA9A gene with Amy signal peptide. The SDS-PAGE shows a representative sample from one of the biological triplicates.

FIG. 8: Stoichiometric quantification of the N-terminal methylation of LsAA9A LPMO, secreted into the medium by K. phaffii cells in the presence or absence of co-expression of NHMT AN4663, using a targeted MS approach. The co-expression of LsAA9A and NHMT AN4663 was performed using biological triplicates. LsAA9A=K. phaffii expressing LsAA9A LPMO; LsAA9A+NHMT=K. phaffii co-expressing NHMT and LsAA9A LPMO. (A) LsAA9A gene with native signal peptide. (B) LsAA9A gene with Amy signal peptide.

FIG. 9: Purification of LPMO LsAA9A and NHMT AN4663 co-expression in K. phaffii. (A) Size exclusion chromatogram of LsAA9A (light grey) and LsAA9A+NHMT (dark grey). Protein elutes at 60 minutes. (B) SDS-page analysis of LsAA9A and LsAA9A+NHMT both show apparent MW of 37 KDa.

FIG. 10: (A) Graphical illustration showing a possible interaction at the ER membrane between the NHMT and the LsAA9A LPMO. It is speculated that the LPMO is first translocated by the Sec translocation machinery of the ER, and once the signal peptide is cleaved, and the LPMO has entered the lumen, the NHMT recognizes and interacts with the LPMO by transferring the methyl group from the S-adenosyl methionine (SAM) onto the LsAA9A. (B) Impact of truncation of AN4663 on activity: PRM quantification of methylated HTVIVPGYR due to truncation of the transmembrane domain of AN4663 (NHMT=AN4663). (C) Impact of truncation of AN4663 on activity: PRM quantification of unmethylated HTVIVPGYR after AN4663 truncation (NHMT=AN4663). (D) Microscopy quantification of AN4663 ER co-localization of different strains as indicated by the Pearson correlation. Error bars indicate the standard error of the mean (NHMT=AN4663). (E) Co-localized image of negative control cytosolic mRFP, positive control endogenous mRFP tagged ER protein C8VRA6, and mRFP-NHMT (N-terminal tagged AN4663) expressing cells. Two different channels: red (mRFP), green (DiCO6), and merged are shown, yellow indicates co-localization in the merged channel.

FIG. 11: Conservation of AN4663. (A) TOPCONS prediction of TMDs (light grey arrows) and NCBI conserved domain search prediction of SAM-dependent methyltransferase domain (black arrow). (B) Alignment of three selected NHMT orthologs and AN4663. TMD region revealed to be less stringent in conservation whereas the catalytic domain in the C-terminus of the protein showed the highest conservation. Conserved residues are distributed throughout the sequence alignment. As an example of key residues, a zoom in on the sequence in the very C-terminus reveal conserved tryptophan/tyrosine residues (with upward-pointing black arrows), which is proposed to be important for the function of the protein.

FIG. 12: (A) Phylogenetic analysis of organisms with sequence similarity to AN4663 7TM (aa residues 1-250). iTOL (Letunic et al 2021) tool was utilized to generate the phylogenetic tree. (B) Alignment of the soluble domain linked to the 452 transmembrane domain sequences by MUSCLE v. 3.8; visualized as sequence motifs in WebLogo 3.0. The region 318-358 shows the methyl transferase motif at 319-323 as well as the SAM binding glutamate in position 340. The highly conserved segment 389-394 is uniquely found in NHMT proteins having the N-terminal 7TM domain.

FIG. 13: Graphical illustration of the process leading to identification of the single NHMT of Aspergillus nidulans.

### ABBREVIATIONS, TERMS, AND DEFINITIONS

Amino acid sequence identity: The term “sequence identity” as used herein, indicates a quantitative measure of the degree of homology between two amino acid sequences of substantially equal length. The two sequences to be compared must be aligned to give a best possible fit, by means of the insertion of gaps or alternatively, truncation at the ends of the protein sequences. The sequence identity can be calculated as ((Nref−Ndif)100)/(Nref), wherein Ndif is the total number of non-identical residues in the two sequences when aligned and wherein Nref is the number of residues in one of the sequences. Sequence identity calculations are preferably automated using the BLAST program e.g. the BLASTP program (Pearson W. R and D. J. Lipman (1988)) (www.ncbi.nlm.nih.gov/cgi-bin/BLAST). Multiple sequence alignment is performed with the sequence alignment method ClustalW with default parameters as described by Thompson J., et al 1994, available at http://www2.ebi.ac.uk/clustalw/.

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3: proline; group 4: Phenylalanine, Tyrosine, Tryptophan; Group 5: Aspartate, Glutamate, Asparagine, Glutamine.

The term genetically engineered gene means a gene that is not native to the cell's genome; or a genetically-engineered derivative of a native gene (e.g. having modified expression controlling elements—such as a non-native promoter and/or RBS sequences); or a native gene that is provided in addition to the native gene already comprised in the host cell's genome.

Native gene: endogenous gene in a microorganism cell genome, homologous to host microorganism.

### DETAILED DESCRIPTION OF THE INVENTION

### I a Genetically-Modified Cell for Production of N-Terminal Histidine Methylated Proteins or Peptides

The invention provides a genetically-modified cell for production of a peptide or polypeptide having a methylated N-terminal histidine residue.

The genetically-modified cell is capable of expressing a first polypeptide having N-terminal histidine methyltransferase activity (NHMT activity). The methyl transferase activity comprises methylation of an N-terminal histidine residue of the polypeptide or peptide. The methyl transferase activity includes methylation of at least one of the nitrogen atoms of the imidazole ring, namely N3 (T-methylation of Nε2) or N1 (or n) of the terminal histidine residue, preferably N3.

The genetically-modified cell is further capable of expressing a peptide or polypeptide having a histidine residue that can be methylated by the first polypeptide having NHMT activity. Said methylation of the peptide or polypeptide includes methylation of at least one of the nitrogen atoms of the imidazole ring, namely N3 (T-methylation of Nε2) or N1 (or n) of the N-terminal histidine residue.

The first polypeptide is encoded by a first nucleic acid sequence comprised in a first gene that is genetically engineered in the genetically-modified cell. In one embodiment the genetically-modified cell comprises a first gene encoding a first polypeptide that is not native to the cell's genome. In an alternative embodiment the first gene encodes a first polypeptide having the same amino acid sequence as a polypeptide encoded by a native gene in the cell's genome, wherein the first gene is either a genetically-engineered derivative of a native gene (e.g. having modified expression controlling elements—such as a non-native promoter and/or RBS sequences) or is provided in addition to said native gene. The first gene comprising the nucleic acid sequence encoding the first polypeptide may be located within the cell's genome or may be located on an episomal vector.

According to one embodiment, the first polypeptide, encoded by the first gene in the genetically-modified cell, is characterized by having a catalytic domain having N-terminal histidine methyltransferase activity. In one embodiment, the catalytic domain employs S-adenosyl-L-methionine as co-substrate and catalyzes the reaction S-adenosyl-L-methionine+protein/peptide having N-terminal histidine <=>S-adenosyl-L-homocysteine+protein or peptide having an N-terminal N3 or N1-methyl-L-histidine.

In one further embodiment, an analog of S-Adenosyl methionine may also be utilized, wherein the methyl group covalently attached to the sulfur atom on SAM is replaced with another chemical moiety such as a dimethyl group, ethyl group, propyl group; and that these groups can be transferred onto methyltransferase enzyme substrate instead of the traditional methyl groups. Thus, in one embodiment, the first polypeptide facilitates transfer of a methyl, dimethyl, ethyl or propyl group to the N-terminal histidine residue of the target peptide or polypeptide.

In one embodiment, the catalytic domain of the first polypeptide is located at the C-terminal region of the polypeptide, said domain being characterized by a SAM binding motif, said motif comprising a glutamate residue essential for its catalytic activity (corresponding to E340 in SEQ ID NO.:2). In one embodiment, the amino acid sequence of the catalytic domain of the first polypeptide has at least 60%, or in order of increasing preference at least 65%, 70%, 71%, 72%, 73%, 74% 75%, 76%, 77%, 78%, 79% 80%, 81%, 82%, 83%, 84% 85%, 86%, 87%, 88%, 89%, 90%, 91%, 92%, 93%, 94% 95%, 96%, 97%, 98%, 99%, or 100% sequence identity to amino acid residues 220 to 558 of SEQ ID NO.: 2.

In a further embodiment, the first polypeptide has an N-terminal region comprising transmembrane spanning domains such that the N-terminal region of the polypeptide is capable of residing in a cellular membrane of the genetically-modified cell. The N-terminal region is one that is capable of anchoring the C-terminal region of the polypeptide to a cellular membrane, while the C-terminal region extends into an aqueous soluble environment, for example into the cytoplasm, or the lumen of the endoplasmic reticulum; or an extracellular space. In one embodiment, the amino acid sequence of the first polypeptide has at least 60%, or in order of increasing preference at least 65%, 70%, 71%, 72%, 73%, 74% 75%, 76%, 77%, 78%, 79% 80%, 81%, 82%, 83%, 84% 85%, 86%, 87%, 88%, 89%, 90%, 91%, 92%, 93%, 94% 95%, 96%, 97%, 98%, 99%, or 100% sequence identity of SEQ ID NO.: 2. The N-terminal region of the polypeptide, corresponding to amino acid residues 1 to 219 of SEQ ID NO.: 2 may comprise at least 2 transmembrane domains, or in order of increasing preference at least 3, 4, 5, 6 or 7 transmembrane domains. By way of example only, the architecture of the membrane-bound first polypeptide having a SAM binding domain and N-terminal histidine methyltransferase activity is illustrated in FIG. 3.

The genetically-modified cell may further comprise a second gene comprising a second nucleic acid sequence encoding peptide or polypeptide, or a precursor thereof, wherein said peptide or polypeptide comprises an N-terminal histidine residue; and wherein said peptide or polypeptide is the target for N-terminal histidine methylation by said first polypeptide. When the target peptide or polypeptide is expressed as a precursor, the target for N-terminal histidine methylation is the N-terminal histidine residue of the mature peptide or polypeptide that results from processing of the precursor, for example removal of an N-terminal signal peptide and/or pro-peptide from the precursor. In a further embodiment, the second gene comprising a second nucleic acid sequence encodes a secreted peptide or polypeptide, said peptide or polypeptide having an N-terminal sequence directing the peptide or polypeptide to the genetically-modified cell's secretory pathway, for example by having an N-terminal signal peptide. While not wishing to be bound by theory, the secretion of a target protein (or peptide); the co-translational removal of its signal peptide; and N-terminal histidine methylation of its N-terminal histidine may occur within the lumen of the ER by the first polypeptide having NHMT activity. The polypeptide or peptide or precursor thereof expressed by the genetically-modified cell of the invention is one that gains an advantage from methylation of the N-terminal histidine residue of the peptide or polypeptide when compared to its corresponding non-methylated form. This advantage may take the form of enhanced stability (for example as measured by half-life), enhanced catalytic activity, enhanced pharmacological activity, enhanced solubility, immobilization, or specificity; enhanced stability/activity with proteases, in alkaline, acidic or saline environment, in the presence of oxidative stress factors, (e.g. H2O2, superoxide, O2, O3), detergents, organic solvents, ionic liquids, or micelle systems.

The peptide or polypeptide encoded by the second nucleic acid sequence comprised in the second gene may be either native or non-native to the genome of the genetically-modified cell. The gene comprising the nucleic acid sequence encoding the peptide or polypeptide, or precursor thereof, may be located within the cell's genome or may be located on an episomal vector.

In one embodiment, the precursor of the target peptide or polypeptide is a secreted peptide or polypeptide having a pre- or a prepro-domain, wherein the pre- or prepro-domain is removed prior to methylation of the N-terminal histidine residue of the target peptide or polypeptide.

The first nucleic acid sequence and the second nucleic acid sequence are each operably linked to a promoter sequence controlling expression of the respective genes. The promoter may be a constitutive promoter or an inducible promoter. In some instances it may be desirable to employ an inducible promoter, particularly for controlling the expression of the second gene comprising the second nucleic acid sequence encoding the peptide, polypeptide or precursor thereof. For example, where the host cell is a eukaryote, such as a species of Saccharomyces, then a suitable inducible promoter is GAL 1 promoter [SEQ ID NO.: 9] or AOX promoter [SEQ ID NO.: 10]. For example, where the host cell is a prokaryote, then a suitable inducible promoter is T7 promoter [SEQ ID NO.: 11]. For example, where the host cell is a mammalian cell, then a suitable promoter is the tetracycline inducible promoter [SEQ ID NO.: 12] or the TET-responsible promoter: CMV (cytomegalovirus minimal promoter) [SEQ ID NO.: 13].

In one embodiment the genetically-modified cell of the invention is a cell that is capable of expressing an increased level of NHMT activity as compared to a parent cell lacking the first gene comprising a nucleic acid sequence encoding a first polypeptide having NHMT activity and from which the genetically-modified cell was derived. In a further embodiment, the genetically-modified cell of the invention is derived from a parent cell that lacks native gene(s) encoding a native fungal S-adenosylmethionine-dependent methyltransferase enzyme.

In one embodiment the genetically-modified cell of the invention is a eukaryotic or prokaryotic cell. The eukaryotic cell may be a mammalian cell (e.g. Chinese Hamster Ovary [CHO] cell, mouse myeloma cell, including an NSO and Sp2/0 cell). The eukaryotic cell may be a microbial cell, preferably belonging to an Ascomycota family; even more preferably a member of the Saccharomycetaceae family. For example the genetically-modified cell can be species of Saccharomyces, K phaffii, Kluyveromyces, Hansenula and Yarrowia. In a preferred embodiment, the genetically modified cells is K phaffii. The prokaryotic cell may be a species of Escherichia, Lactobacillus, Bacillus, Brevibacterium, Corynebacterium, Mycobacterium, Nocardia, Streptomyces, Chromohalobacter, Halomonas, Pseudomonas, Shewanella, Rodhobacter and Caulobacter. In one embodiment, the genetically-modified cell of the invention comprises a first polypeptide having N-terminal histidine methyltransferase activity (NHMT activity) encoded by a first nucleic acid sequence comprised by a first gene and a peptide or protein or precursor thereof encoded by a second nucleic acid sequence comprised by a second gene, wherein said peptide or protein has a methylated N-terminal histidine residue; and wherein said methylation comprises at least one of the nitrogen atoms of the imidazole ring, namely N3 (T-methylation of Nε2) or N1 (or n) of the terminal histidine residue.

In one aspect, the invention provides a genetically-modified cell for production of a target peptide or polypeptide having a modified N-terminal histidine residue, wherein the cell comprises:
a. a first gene comprising a first nucleic acid sequence encoding a first polypeptide exhibiting N-terminal histidine methyltransferase activity, wherein said first gene is genetically engineered,
   wherein said first polypeptide comprises an amino acid sequence having at least 60% sequence identity to amino acid residues 220 to 558 of SEQ ID NO.: 2, and
b. a second gene comprising a second nucleic acid sequence encoding a target peptide or polypeptide, or a precursor thereof,
   wherein said target peptide or polypeptide comprises a N-terminal histidine residue; and
   wherein said first polypeptide facilitates modification of the N-terminal histidine residue of the target peptide or polypeptide.

In one embodiment, said modification of the N-terminal histidine residue of the target peptide or polypeptide comprises transfer of a methyl, dimethyl, ethyl or propyl group to the N-terminal histidine residue of the target peptide or polypeptide. In a preferred embodiment, the N-terminal histidine residue of the target peptide or polypeptide is methylated.

In one embodiment, the precursor of said target peptide or polypeptide is a secreted peptide or polypeptide having a pre- or a prepro-domain, and wherein the pre- or prepro-domain is removed prior to modification of the N-terminal histidine residue of the target peptide or polypeptide.

### II. A Genetically-Modified Cell for Production of N-Terminal Histidine Methylated LPMO

In one embodiment the genetically-modified cell of the invention has a first gene comprising a first nucleic acid encoding a polypeptide having NHMT activity and a second gene comprising a second nucleic acid sequence encoding a Lytic Polysaccharide Monooxygenase (LPMO; alternative names include PMO, GH61 or CBP21). LPMOs that are classified in CAZy as auxiliary activity (AA9-AA11, AA13-AA17) possess a distinct catalytic site, comprised of a His-pair coordinating a copper ion. One of the two catalytic histidine residues is located at the N-terminus and the α-amino group of the N-terminal Histidine takes part in the active site copper. Many native LPMOs secreted by filamentous fungi comprise an N-terminally methylated histidine residue, particularly LPMOs that are classified in CAZy as auxiliary activity AA9 and AA13.

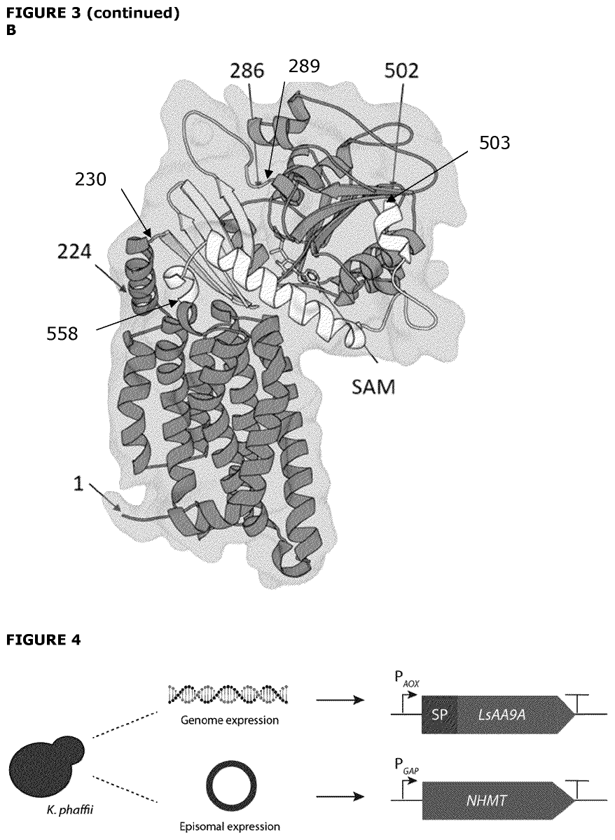

LPMO encoded by the second nucleic acid sequence comprises a signal peptide that co-translationally targets synthesis of the LPMO to the endoplasmic reticulum (ER) and its transport into the ER lumen. Recognition and correct cleavage of the signal peptide of LPMO, and methylation of the N-terminal histidine of the mature LPMO (revealed by signal peptide cleavage) is important for its activity and/or stability. The signal peptide sequence encoded by the second nucleic acid sequence is chosen according to the chosen host employed as genetically-modified cell. Accordingly, the signal peptide may be the native LPMO signal peptide, or may be a heterologous signal peptide. Co-expression of a polypeptide having NHMT activity with an AA9 LPMO polypeptide having a native or heterologous signal peptide in a genetically-modified cell of the invention is exemplified in FIGS. 4-7. Here the cells are shown to secrete mature LPMO having an N-terminally methylated histidine residue due to correct signal peptide cleavage and methylation by the co-expressed NHMT.

Examples of AA9 LPMOs include LsAA9A from Lentinus similis (Genbank accession number: CAD21296.1) [SEQ ID NO.: 15], NcLPMO9 from Neurospora crassa (Genbank accession number: CAD21296.1) [SEQ ID NO.: 17], HrLPMO9 from Heterobasidion irregulare (Genbank accession number: ETW87087.1) [SEQ ID NO.: 19], TtLPMO9A from Thermothelomyces thermophilus (Genbank accession number: AKO82493) [SEQ ID NO: 21], PaLPMO9E from Podospora anserine (Genbank accession number: CAP67740.1) [SEQ ID NO.: 23], and PaLPMO9H from Podospora anserina (Genbank accession number: CAP61476.1) [SEQ ID NO.: 25].

### III. A Genetically-Modified Cell for Production of N-Terminal Histidine Methylated Incretin Hormone

In one embodiment the genetically-modified cell of the invention has a first gene comprising a first nucleic acid encoding a polypeptide having NHMT activity and a second gene comprising a second nucleic acid sequence encoding an incretin peptide hormone. One member of this class of hormone is Glucagon-like peptide-1 (GLP-1), whose native gene encodes preproglucagon, which is post-translationally cleaved to release GLP-1 having an N-terminal histidine. The active secreted forms of GLP-1 derived from the precursor GLP-1, are GPP-1 (7-36) amide and GLP-1(7-37). According to one embodiment, the first nucleic acid encodes a precursor GLP-1 comprising a heterologous N-terminal signal peptide fused to GLP-1(7-36) [SEQ ID NO.: 27] or GLP-1(7-37) [SEQ ID NO.: 29]. Suitable signal peptides include native signal peptide of the LPMO [SEQ ID NO.: 31], OST1 [SEQ ID NO.: 33], AmySP [SEQ ID NO.: 35]. A suitable integration vector for insertion of the GLP-1 (or analogue thereof) encoding gene into K. phaffii includes pPIC9K as an integrative vector for suitable expression of LPMOs

Further members of this class of hormone are analogues of Glucagon-like peptide-1 (GLP-1) For example the GLP-1 analogue having SEQ ID NO.: 37 (Lixisenatide); GLP-1 analogue having SEQ ID NO.: 39 (Exenatide); GLP-1 analogue having SEQ ID NO.: 41 (Liraglutide); GLP-1 analogue having SEQ ID NO.: 43 (Albiglutide); GLP-1 analogue having SEQ ID NO.: 45 (Dulaglutide); GLP-1 analogue having SEQ ID NO.: 46 (Semaglutide).

In some embodiments, the first nucleic acid encodes a precursor comprising a heterologous N-terminal signal peptide fused to GLP-1, or analogue thereof, wherein the GLP-1, or analogue thereof is further fused to a second protein such as Human Serum Albumin (HSA) or an immunoglobulin fragment, preferably to its C-terminus. Production of an N-terminal histidine methylated incretin hormone is exemplified in Example 7.

### IV. An Isolated Histidine N-Methyltransferase Enzyme

In one aspect, the present invention provides an isolated polypeptide having NHMT activity for catalyzing the reaction S-adenosyl-L-methionine+protein/peptide having N-terminal histidine <=>S-adenosyl-L-homocysteine+protein or peptide having an N-terminal N3 or N1-methyl-L-histidine.

In one embodiment, the isolated polypeptide corresponds to the catalytic domain of a polypeptide having at least 60%, or in order of increasing preference at least 65%, 70%, 71%, 72%, 73%, 74% 75%, 76%, 77%, 78%, 79% 80%, 81%, 82%, 83%, 84% 85%, 86%, 87%, 88%, 89%, 90%, 91%, 92%, 93%, 94% 95%, 96%, 97%, 98%, 99%, or 100% sequence identity to amino acid residues 220 to 558 of SEQ ID NO.: 2.

In a further embodiment, the isolated polypeptide corresponds to a polypeptide having at least 60%, or in order of increasing preference at least 65%, 70%, 71%, 72%, 73%, 74% 75%, 76%, 77%, 78%, 79% 80%, 81%, 82%, 83%, 84% 85%, 86%, 87%, 88%, 89%, 90%, 91%, 92%, 93%, 94% 95%, 96%, 97%, 98%, 99%, or 100% amino acid sequence identity to SEQ ID NO.: 2.

In one embodiment, the isolated polypeptide corresponds to the catalytic domain of a polypeptide having at least 60%, or in order of increasing preference at least 65%, 70%, 71%, 72%, 73%, 74% 75%, 76%, 77%, 78%, 79% 80%, 81%, 82%, 83%, 84% 85%, 86%, 87%, 88%, 89%, 90%, 91%, 92%, 93%, 94% 95%, 96%, 97%, 98%, 99%, or 100% sequence identity to amino acid residues 302 to 497 of SEQ ID NO.: 4 (homolog from Aspergillus fumigatus).

In a further embodiment, the isolated polypeptide corresponds to a polypeptide having at least 60%, or in order of increasing preference at least 65%, 70%, 71%, 72%, 73%, 74% 75%, 76%, 77%, 78%, 79% 80%, 81%, 82%, 83%, 84% 85%, 86%, 87%, 88%, 89%, 90%, 91%, 92%, 93%, 94% 95%, 96%, 97%, 98%, 99%, or 100% amino acid sequence identity to SEQ ID NO.: 4 (homolog from Aspergillus fumigatus).

In one embodiment, the isolated polypeptide corresponds to the catalytic domain of a polypeptide having at least 60%, or in order of increasing preference at least 65%, 70%, 71%, 72%, 73%, 74% 75%, 76%, 77%, 78%, 79% 80%, 81%, 82%, 83%, 84% 85%, 86%, 87%, 88%, 89%, 90%, 91%, 92%, 93%, 94% 95%, 96%, 97%, 98%, 99%, or 100% sequence identity to amino acid residues 353 to 497 of SEQ ID NO.: 6 (homolog from Neurospora crassa).

In a further embodiment, the isolated polypeptide corresponds to a polypeptide having at least 60%, or in order of increasing preference at least 65%, 70%, 71%, 72%, 73%, 74% 75%, 76%, 77%, 78%, 79% 80%, 81%, 82%, 83%, 84% 85%, 86%, 87%, 88%, 89%, 90%, 91%, 92%, 93%, 94% 95%, 96%, 97%, 98%, 99%, or 100% amino acid sequence identity to SEQ ID NO.: 6 (homolog from Neurospora crassa).

In one embodiment, the isolated polypeptide corresponds to the catalytic domain of a polypeptide having at least 60%, or in order of increasing preference at least 65%, 70%, 71%, 72%, 73%, 74% 75%, 76%, 77%, 78%, 79% 80%, 81%, 82%, 83%, 84% 85%, 86%, 87%, 88%, 89%, 90%, 91%, 92%, 93%, 94% 95%, 96%, 97%, 98%, 99%, or 100% sequence identity to amino acid residues 385 to 496 of SEQ ID NO.: 8 (homolog from Neolentinus lepideus).

In a further embodiment, the isolated polypeptide corresponds to a polypeptide having at least 60%, or in order of increasing preference at least 65%, 70%, 71%, 72%, 73%, 74% 75%, 76%, 77%, 78%, 79% 80%, 81%, 82%, 83%, 84% 85%, 86%, 87%, 88%, 89%, 90%, 91%, 92%, 93%, 94% 95%, 96%, 97%, 98%, 99%, or 100% amino acid sequence identity to SEQ ID NO.: 8 (homolog from Neolentinus lepideus).

The respective isolated polypeptides are both characterized by a SAM binding motif or domain, said motif/domain comprising a glutamate residue essential for its catalytic activity (corresponding to E340 in SEQ ID NO.: 2).

Production of the catalytic domain of NHMT is exemplified in Example 7. Production of the full-length NHTM protein (AN4663) including its transmembrane domain can be performed by transforming a suitable NHMT protein expression vector into a suitable host cell, such as E. coli. After protein production, extraction, and purification of the membrane bound-produced AN4663 can be performed using detergents such as N-Dodecyl b-D-maltoside to aid solubilization.

When the isolated polypeptides having NHMT activity are brought into contact with a peptide or protein having an N-terminal histidine, the peptide or protein is N-terminally methylated, as described in Example 7.

In an in-vitro setting, a purified fraction of the histidine N-methyltransferase enzyme (such as only the catalytic histidine N-methyltransferase domain or the catalytic histidine N-methyltransferase domain fused to other protein, biomolecules, and/or chemical moieties, i.e. to increase stability) can be incubated with the donor S-Adenosyl methionine (also referred to as “AdoMet” or “SAM”) containing a methyl group that is to be transferred, and target substrate (such as a peptide, protein, incretin hormone, or anything containing an free amine (NH2) followed by a histidine, followed by subsequent sequence of amino acids, small molecule, or other chemical groups).

An analog of S-Adenosyl methionine may also be utilized, wherein the methyl group covalently attached to the sulfur atom on SAM is replaced with another chemical moiety such as a dimethyl group, ethyl group, propyl group; and that these groups can be transferred onto methyltransferase enzyme substrate instead of the traditional methyl groups.

Furthermore, the methyl group can be replaced with reactive groups in order to facilitate site selective reaction on n-terminal histidines. These reactive groups can be a number of functional groups such as those containing an azide or alkyne group, typically utilized in combination for specific cycloaddition chemistry limiting side reactions and products, also referred to as “click chemistry” (Hartmuth et al 2001). These reactive groups can be utilized to perform site specific chemistry or bioconjugation to form chemical links with other chemical and biomolecular species such as small molecules, drugs, proteins, peptides, or living cells and organisms. This in turn could be utilized to change the chemical and biological properties of the histidine modified molecules for advantages purposes such as increased bioavailability, increased or decreased biological half-life, and if a functional protein or antibody, to increase selectivity and specificity; it may also be utilized to selectively deliver small molecule drugs to an intended biological target, this target could be, but not limited to, protein, cell, or organ specific.

### V. Method for Identifying a Histidine N-Methyltransferase Enzyme

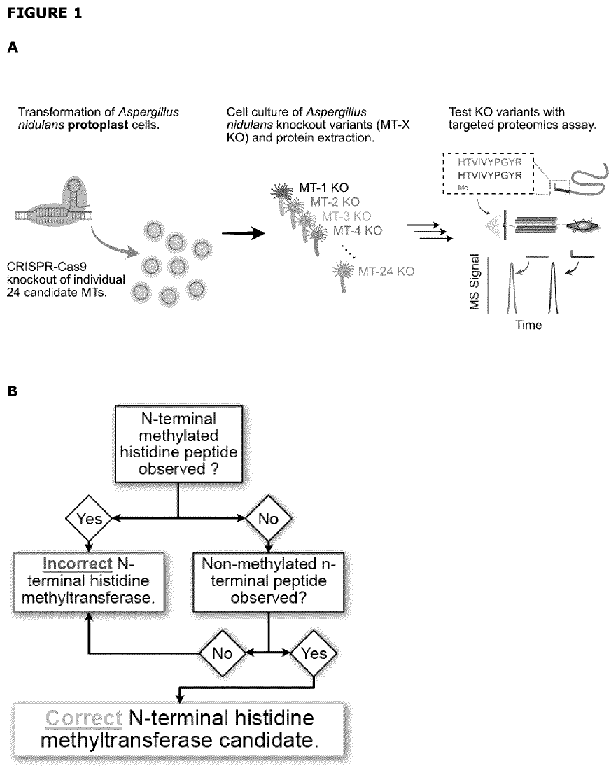

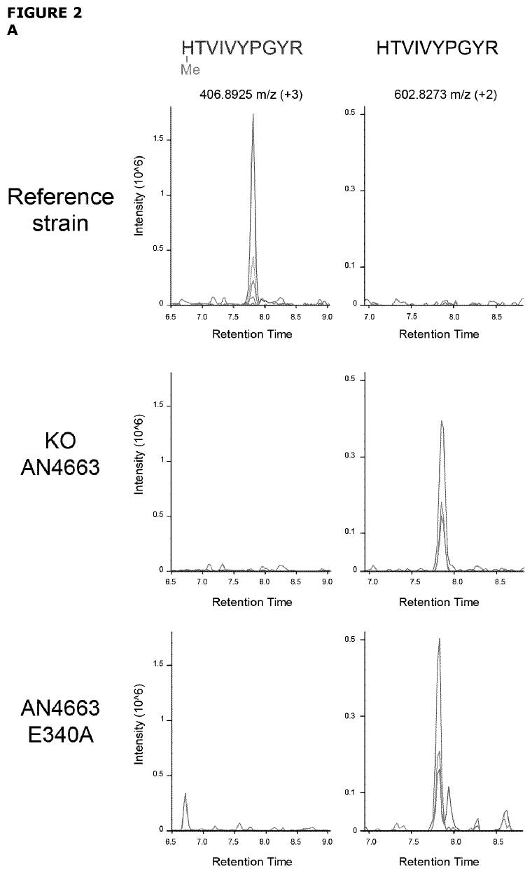

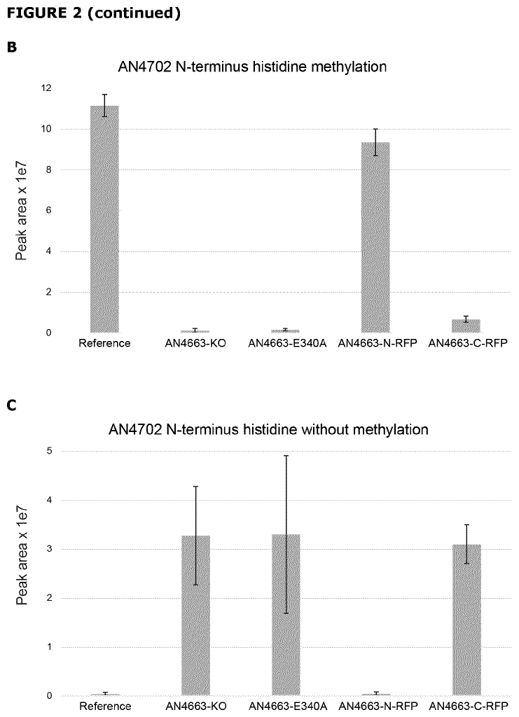

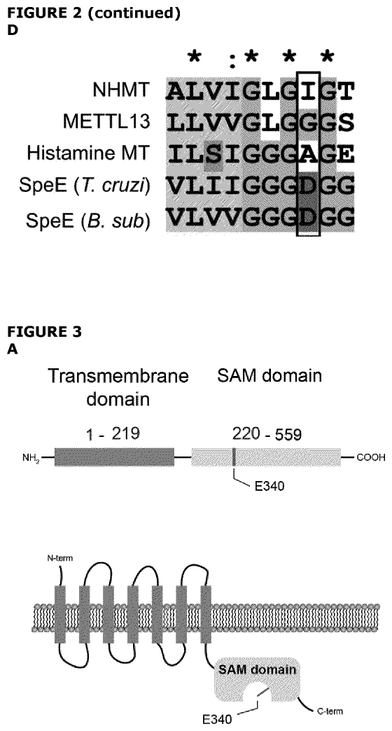

A candidate polypeptide can be assayed for NHMT activity by cloning and expressing a gene encoding the polypeptide in a suitable host cell that is itself devoid of genes encoding an active NHMT, for example a S. cerevisiae cell. Alternatively the candidate polypeptide can be expressed from a native gene in its native host. The host cell expressing the candidate polypeptide may be cultured; the whole proteome can then be extracted and then subjected to a targeted proteomics assay (PMR) to detect N-terminal methylation of a synthetic peptide, as described in Example 1, and illustrated in FIGS. 1 and 2.

### VI. Methods for Preparing an N-Terminal Histidine Methylated Polypeptide or Peptides In Vivo

In one aspect, the invention provides a method for production of a target peptide or polypeptide having a modified N-terminal histidine residue, comprising the steps of:
a. providing a genetically-modified cell according to the invention, or a cell population derived therefrom;
b. culturing the cell or cell population under conditions allowing expression of the first gene comprising a first nucleic acid sequence encoding a first polypeptide exhibiting N-terminal histidine methyltransferase activity and the second gene comprising a second nucleic acid sequence encoding a target peptide or polypeptide, or a precursor thereof,
c. recovering the target peptide or polypeptide having a modified N-terminal histidine residue.

In one embodiment, the target peptide or polypeptide is modified by transfer of a methyl, dimethyl, ethyl or propyl group to the N-terminal histidine residue of the target peptide or polypeptide. In a preferred embodiment, the target peptide or polypeptide is methylated.

In one aspect the invention provides a method for production of a target peptide or polypeptide having a methylated N-terminal histidine residue, said method comprising the steps of:
a. providing a genetically-modified cell according to the invention (as described in section I), or a cell population derived therefrom;
b. culturing the cell or cell population under conditions allowing co-expression of the the first gene comprising a first nucleic acid sequence encoding the first polypeptide having NHMT activity and the second gene comprising the second nucleic acid sequence encoding a target peptide or polypeptide or a precursor thereof,
c. recovering the target peptide or polypeptide having a methylated N-terminal histidine residue.

In one embodiment the target polypeptide or its precursor is LPMO, preferably an AA9 LPMO (as described in section II). In a further embodiment the target peptide is an incretin hormone, for example GLP-1, (as described in section III).

In one embodiment the genetically-modified cell of the invention is a eukaryotic or prokaryotic cell. The eukaryotic cell may be a mammalian cell (e.g. Chinese Hamster Ovary [CHO] cell, mouse myeloma cell, including an NSO and Sp2/0 cell). The eukaryotic cell may be a microbial cell, preferably belonging to an Ascomycota family; even more preferably a member of the Saccharomycetaceae family. For example the genetically-modified cell can be species of Saccharomyces, K. phaffii, Kluyveromyces, Hansenula and Yarrowia. In a preferred embodiment, the genetically modified cells is K phaffii. In a preferred embodiment, the genetically modified cells is a species of Saccharomyces. The prokaryotic cell may be a species of Escherichia, Lactobacillus, Bacillus, Brevibacterium, Corynebacterium, Mycobacterium, Nocardia, Streptomyces, Chromohalobacter, Halomonas, Pseudomonas, Shewanella, Rodhobacter and Caulobacter.

### VII. Use of Genetically-Modified Cell of the Invention for Preparing an N-Terminal Histidine Methylated Polypeptide or Peptide In Vivo

A genetically-modified cell of the invention may be used to produce a peptide or polypeptide with an N-terminally modified histidine, wherein the N-terminal modification comprises modification of the N3 or N1 nitrogen atom of the histidine. The modification, catalyzed by the first polypeptide having NHMT activity, is the result of the transfer of any one of a methyl group, dimethyl group, ethyl group, or propyl group to the N3 or N1 nitrogen atom of the histidine. The methylated polypeptide or peptide includes those described in section II and III.

In one aspect, the invention provides the use of the genetically-modified cell according to the invention or the isolated polypeptide according to the invention, for production of a target peptide or polypeptide having a modified N-terminal histidine residue. In one embodiment, said modification of the N-terminal histidine residue comprises methylation, dimethylation, ethylation or propylation. In a preferred embodiment, said N-terminal histidine residue is methylated.

### Preferred Numbered Embodiments of the Invention

Preferred numbered embodiment 1. A genetically-modified cell for production of a target peptide or polypeptide having a methylated N-terminal histidine residue, wherein the cell comprises:
a. a first gene comprising a first nucleic acid sequence encoding a first polypeptide exhibiting N-terminal histidine methyltransferase activity,
   wherein said first gene is genetically engineered,
   wherein said first polypeptide comprises an amino acid sequence having at least 60% sequence identity to amino acid residues 220 to 558 of SEQ ID NO.: 2, and
b. a second gene comprising a second nucleic acid sequence encoding a target peptide or polypeptide, or a precursor thereof,
   wherein said target peptide or polypeptide comprises a N-terminal histidine residue; and
   wherein said first polypeptide facilitates methylation of the N-terminal histidine residue of the target peptide or polypeptide.

Preferred numbered embodiment 2. The genetically-modified cell according to preferred numbered embodiment 1, wherein said first polypeptide further comprises an N-terminal transmembrane domain.

Preferred numbered embodiment 3. The genetically-modified cell according to preferred numbered embodiment 1 or 2, wherein the amino acid sequence of the first polypeptide has at least 60% sequence identity to SEQ ID NO.: 2.

Preferred numbered embodiment 4. The genetically-modified cell according to any one of preferred numbered embodiments 1 to 3, wherein the first polypeptide is an S-adenosylmethionine-dependent methyltransferase.

Preferred numbered embodiment 5. The genetically-modified cell according to any one of preferred numbered embodiments 1 to 4, wherein
a. the first gene comprising a first nucleic acid sequence encodes a first polypeptide that is not native to said cell, and/or
b. the second gene comprising a second nucleic acid sequence encoding the target peptide or polypeptide is genetically engineered and not native to said cell.

Preferred numbered embodiment 6. The genetically-modified cell according to any one of preferred numbered embodiments 1 to 5, wherein the precursor of said target peptide or polypeptide is a secreted peptide or polypeptide having a pre- or a prepro-domain, and wherein the pre- or prepro-domain is removed prior to methylation of the N-terminal histidine residue of the target peptide or polypeptide.

Preferred numbered embodiment 7. The genetically-modified cell according to any one of preferred numbered embodiments 1 to 6, wherein:
a. the target polypeptide is a lytic polysaccharide monooxygenase, and
b. the target peptide is an incretin hormone.

Preferred numbered embodiment 8. The genetically-modified cell according preferred numbered embodiments 7, wherein the incretin hormone is glucagon-like peptide-1 (GLP-1) or an analogue thereof, wherein GLP-1 or the analogue is selected from the group having an amino acid sequence of SEQ ID NO.: 37, 39, 41, 43, 45, and 46.

Preferred numbered embodiment 9. The genetically-modified cell according to any one of preferred numbered embodiments 1-8, wherein the genetically-modified cell is selected from K. phaffii and a species of Saccharomyces.

Preferred numbered embodiment 10. An isolated polypeptide exhibiting histidine N-methyltransferase enzyme activity,
wherein said polypeptide comprises an amino acid sequence having at least 60% sequence identity to amino acid residues 220 to 558 of SEQ ID NO.: 2.

Preferred numbered embodiment 11. A method for production of a target peptide or polypeptide having a methylated N-terminal histidine residue, comprising the steps of:
a. providing a genetically-modified cell according to any one of preferred numbered embodiments 1-9, or a cell population derived therefrom;
b. culturing the cell or cell population under conditions allowing expression of the first gene comprising a first nucleic acid sequence encoding a first polypeptide exhibiting N-terminal histidine methyltransferase activity and the second gene comprising a second nucleic acid sequence encoding a target peptide or polypeptide, or a precursor thereof,
c. recovering the target peptide or polypeptide having a methylated N-terminal histidine residue.

Preferred numbered embodiment 12. A method according to preferred numbered embodiment 11, wherein said target polypeptide or precursor thereof is a lytic polysaccharide monooxygenase.

Preferred numbered embodiment 13. A method according to preferred numbered embodiment 11, wherein said target peptide or precursor thereof is an incretin hormone.

Preferred numbered embodiment 14. Use of the genetically-modified cell according to any one of preferred numbered embodiments 1 to 9 or the isolated polypeptide according to claim 10, for production of a target peptide or polypeptide having a methylated N-terminal histidine residue.

Preferred numbered embodiment 15. The use according to preferred numbered embodiment 14, wherein
a. the target polypeptide is a lytic polysaccharide monooxygenase, and
b. the target peptide is an incretin hormone.

### Examples

### General Methodology

### Host Cell Cultivation

Production of spores for inoculations and validation of genetically modified host cells were carried out in liquid or solid (2% agar) minimal medium (MM; 1% glucose, 1× nitrate salt solution40, 0.001% Thiamine, 1× trace metal solution (Cove et al 1966), which was supplemented 4 mM L-arginine (arg) when required. In addition, liquid PD, and cellulose-enriched MM were applied for proteome analysis.

### Protoplastation and Transformation

Protoplastation was performed as described in (Nielsen et al 2006), except for glucanex which was substituted for Glucanex MG (kind gift from Novozymes A/S). The transformations were made in gently thawed protoplasts as described in (Nødvig et al 2018). After transformation, strains were incubated at 37 degrees and afterward validated by diagnostic PCR as described in (Nødvig et al 2015).

### List of Strains and Plasmids

TABLE 1
A. nidulans strains

| Strain ID | Modified site(s) | Genotype |
|---|---|---|
| NID2531 | | argB2, veA1, nkuAΔ |
| NID2787 | AN4663 | argB2, veA1, nkuAΔ, AN4663::E340A:: |
| NID2713 | AN4663 | argB2, veA1, nkuAΔ, AN4663Δ |
| NID2789 | AN4663 & IS5 | argB2, veA1, nkuAΔ, AN4663Δ, IS5::Ptef-N-tag- |
| | | mRFP-AN4663-Ttef:: |
| NID2794 | AN4663 & IS5 | argB2, veA1, nkuAΔ, AN4663Δ, IS5::Ptef-AN4663- |
| | | C-tag-mRFP-Ttef:: |

IS5 = insertion site in A. nidulans genome

TABLE 2
E. coli and K. phaffi parent and genetically modified derivatives

| Host cell | Genotype | Source |
|---|---|---|
| E. coli NEB5α | fhuA2 Δ(argF-lacZ)U169 phoA glnV44 Φ80 | a |
| | Δ(lacZ)M15 gyrA96 recA1 relA1 endA1 thi-1 | |
| | hsdR17 | |
| E. coli BL21(DE3) | F⁻ ompT gal dcm lon hsdS_{B}(r_{B}⁻m_{B}⁻) λ(DE3 | b |
| | [lacI lacUV5-T7p07 ind1 sam7 nin5]) | |
| | [malB⁺]_{K-12}(λ^{S}) | |
| K. phaffii (P. pastoris) | his4 | c |
| K. phaffii Native^{SP}- | aox1::P_{AOX1}-Native^{SP}-LsAA9A-His6-HIS4 + | This |
| LsAA9A Mut⁺ | episomal plasmid-AN4663 | study |
| K. phaffii Amy^{SP}- | aox1::P_{AOX1}-Amy^{SP}-LsAA9A-His6-HIS4 + | |
| LsAA9A Mut⁺ | episomal plasmid-AN4663 | |

TABLE 3
Plasmids

| Plasmid | Description | Source |
|---|---|---|
| pLyGo-Ec-1- | Vector encoding a ubiquitin solubility tag and the | This |
| cytosolicAN4663 | cytosolic domain of AN4663, Km^{R} | study |
| [SEQ ID NO.: 49] | | |
| pLyGo-Kp-Native^{sp}- | Integrative vector into the AOX1 locus. Encodes | This |
| LsAA9A | LsAA9A together with its native signal peptide for | study |
| [SEQ ID NO.: 50] | secretion. Km^{R} (E. coli), HIS4 (K. phaffii) | |
| pLyGo-Kp-Amy^{sp}- | Integrative vector into the AOX1 locus. Encodes | 2 |
| LsAA9A | LsAA9A together with Amy signal peptide for | |
| [SEQ ID NO.: 51] | secretion. Km^{R} (E. coli), HIS4 (K. phaffii) | |
| Episomal plasmid | Episomal vector with a pGAP constitute promoter, | — |
| | Amp^{R} (E. coli), Zeocin^{R} (K. phaffii) | |
| pFC331 | CRISPR vector with Cas9, Amp^{R} (E. coli), ORI, ArgB | 1 |
| | (A. nidulans), AMA1. | |
| pLyGo-Kp-2 | Integrative vector into the AOX1 locus. Encodes the | 2 |
| [SEQ ID NO.: 52] | Amy signal peptide for secretion. Km^{R} (E. coli), HIS4 | |
| | (K. phaffii) | |

1. Nødvig et al. 2018; 2. Hernández-Rollán et al. 2021.

### Example 1: Shortlisting Aspergillus nidulans N-Terminal Histidine Methylation Candidates

According various databases such as Interpro, pfam, and Prosite, there can be up to (but not limited to) 225 predicted putative methyltransferase genes in the Aspergillus nidulans proteome.

Differential proteomics analysis of A. nidulans cells grown on cellulose or glucose containing media was used to narrow down the list of candidates which could be N-terminal Histidine Methyltransferases (NHMT). It was reasoned that a NHMT candidate would be co-expressed with LPMOs specifically when grown in conditions where its activity is needed, for instance when A. nidulans is grown on cellulose, since cellulose is a primary carbon source requiring LPMO activity for it to be utilized by A. nidulans.

A large-scale quantitative proteomics screen was performed by analyzing differentially expressed proteins of cells grown on the different carbon sources. This was accomplished through single shot label free quantification (LFQ) and tandem mass tag (TMT) labeled multiplex in-depth quantitative mass spectrometry-based analysis. Additionally, non-quantitative label free deep proteome sequencing was performed for A. nidulans.

To identify NHMT candidates, relative protein abundances between cells grown on glucose media (including potato dextrose) was compared with cells grown on cellulose, focusing on identifying methyltransferases which displayed differential abundances between the different growth conditions. Methyltransferases were marked by a combination of Interpro (Blum et al 2021), pfam (Mistry et al 2021), PROSITE (Sigrist et al 2012), and gene ontology annotations. Ultimately, 120 of the 225 predicted methyltransferases in the A. nidulans proteome were identified, of which 41 were found to have statistically significant higher abundance when A. nidulans was grown on cellulose as the primary carbon source compared to glucose or potato dextrose. Seven of these 41 methyltransferases could be eliminated from the candidate list due to their prediction as methyltransferases with oxygen as the acceptor atom and not nitrogen. Moreover, phylogenetic analysis revealed that 29 of the remaining 34 methyltransferases appeared to be unique among fungi known to encode for LPMOs. Consequently, when filtering out orthologs in non-LPMO N-terminal histidine methylating organisms, 24 candidates with possible methyltransferase activity were left on the list.

### Example 2: CRISPR/Cas9 Knockout Screen of Methyltransferase Candidates Coupled with Targeted MS Analysis of LPMO Methylation Status

To identify the hitherto unknown methyltransferase (MTase) responsible for N-terminal methylation of LPMOs in filamentous fungi, a systematic CRISPR/Cas9 knockout screen was performed in Aspergillus nidulans of the 24 candidates to identify the putative NHMT candidate.

### 2.1 CRISPR/Cas9-Mediated Engineering

A. nidulans [NID2531] was used as the reference for all knockouts and subsequent studies. It contains the mutation or deletion for 3 genes: Ornithine transcarbamylase (argB2) of the arginine biosynthesis pathway, which is utilized as a selection pressure for transformations via auxotrophic growth requirement of arginine, (veA1Δ) which suppresses the ability of A. nidulans for sexual reproduction, and lastly nkuAΔ which eliminate the non-homologous end-joining repair mechanism and forces the repairing to be through homologous recombination (Nødvig et al 2018).

To precisely and efficiently knock out each of the 24 genes by CRISPR/Cas9, guide RNAs were designed for each candidate and a Cas9-expressing A. nidulans derivative deficient in the error-prone non-homologous end-joining DNA repair was used to ensure high-fidelity genome editing through homologous recombination. 24 knockout strains were systematically generated by deleting each candidate in turn.

All deletions and codon substitutions in A. nidulans were enabled by the oligonucleotide mediated gene-editing procedure described in (Nødvig et al 2018) except for the total length of the GE-oligos being 60 nt for both deletions and the point mutation introductions.

### 2.2 PRM Assay

To analyze the effect on N-terminal histidine methylation of the individual knockouts, a novel targeted proteomics parallel reaction monitoring (PRM) assay was developed to specifically monitor and quantify the native N-terminally histidine methylated A. nidulans peptide sequences identified with the deep proteomics approach described above. In a typical PRM assay, a peptide of interested is isolated by the mass spectrometer (from its mass to charge or m/z value) and fragmented to generate peptide sequence specific fragments such as y- or b-ions, which are predictable depending on the peptide sequence and any sequence modifications. These fragments are specific for the peptide sequence, thus increasing the specificity and sensitivity of the assay. The mass spectrometer is instructed to continuously isolate the peptide of the desired mass to charge (i.e. the methylated and unmethylated n-terminal histidine containing peptide) followed by fragmentation of the said peptide, generating sequence specific y- or b-series ions. All resulting ions were than analyzed by a high-resolution and high-precision mass spectrometer (such as those, but not limited to, based on the Orbitrap mass analyzer technology). If the predicted fragment ions based on the isolated peptide mass are detected, the peptide can be identified and quantified, by elution of the peptide from the column and analysis in a mass spectrometer. The instrument is set to cycle through the given peptide list and to fragment the peptide to generate sequence specific ions rapidly (multiple times a second), generating an elution profile of the fragment ions until the peptide is eluted off the column and enters the mass spectrometer. Consequently, multiple overlapping peptide specific fragments will be detected of a period of time when the peptide elutes off the column. Summing the peak area of fragment peaks can be used for quantitation and detection.

To analyze the effect on N-terminal histidine methylation of the individual knockouts, a novel targeted proteomics parallel reaction monitoring (PRM) mass spectrometry based assay was used to specifically to monitor and quantify a native n-terminally histidine methylated A. nidulans peptide sequences of the protein AN4702 (uniprot entry Q5B428). The sequence of the n-terminal peptide sequence monitored by the mass spectrometer was “HTVIVYPGYR” [SEQ ID NO.: 57]. The mass of the peptide without any charges was 1204.38 g/mol, however with two positive charges, the mass over charge ratio (that was detected by the mass spectrometer) becomes 602.8273 m/z. Similarly, the mass of the histidine methylated version of the same peptide ([methy] HTVIVYPGYR [SEQ ID NO.: 58]) is 1218.4 g/mol and mass to charge ratio of 406.89 m/z when the peptide contains 3 positive charges. The m/z values for the methylated (602.8273 m/z) and unmethylated (406.89 m/z) version of the peptides were selected for PRM analysis by the mass spectrometer. The reason for monitoring different charge states is due to the ionization properties of peptides, certain peptide charge states ionize better in the mass spectrometer (which results in higher sensitivity), and the highest charge state version of the peptide were selected for both peptides in order to ensure highest sensitivity. For a MTase knockout to be considered a possible n-terminal histidine methyltransferase candidate, the loss of n-terminal histidine methylated peptide of AN4702 must be observed and the detection of the counter unmethylated version of the peptide must be detected (FIG. 2). This was only observed with the knockout of the methyltransferase AN4663.

This quantitative PRM analysis revealed that 23 of 24 methyltransferase knockouts did not have any effect on the methylation state of the protein (AN4702). However, the knockout of the gene [SEQ ID NO.: 1] encoding AN4663 (uniprot entry Q5B467) [SEQ ID NO.: 2] abolished the N-terminal histidine methylation on the monitored peptides and conversely, the unmethylated form was observed (FIGS. 2 A, B, and C).

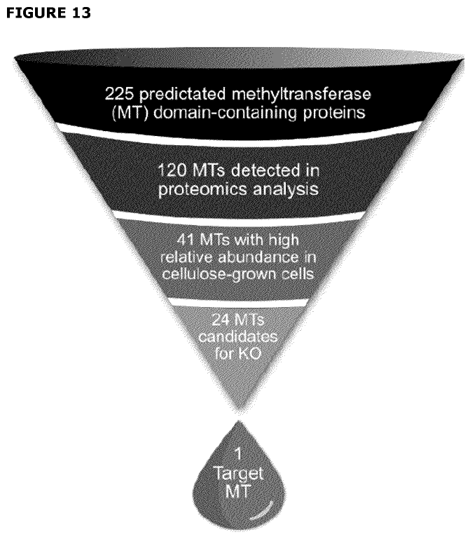

FIG. 13 shows a graphical illustration of the process; starting with the 225 putative methyl transferases identified in Aspergillus nidulans; shortlisting to 120 NHMT candidates by differential proteomics analysis, of which 41 were found to have statistically significant higher abundance when A. nidulans was grown on cellulose as the primary carbon source compared to glucose or potato dextrose; further performing gene knockout experiments to identify the single NHMT.

### Example 3: AN4663

### 3.1 Structural Analysis

In-silico structural analysis of the 558 amino acids long AN4663 protein using PSIPRED (McGuffin et al 2000) and transmembrane protein topology prediction using MEMSAT (Torlak et al 2010) revealed a unique domain architecture with a predicted N-terminal seven-transmembrane domain up to amino acid (AA) position 219 followed by a soluble methyltransferase containing domain at the C-terminal (FIG. 3A).

An AlphaFold2 predicted structural model of NHMT includes a Rossmann-like domain, a beta sheet rich region, and a unique C-terminal extension that wraps around the ectodomain that is ultimately buried in the transmembrane domains (FIG. 3B). The structural model predicts AN4663 to contain an intermediate sized substrate-binding cavity required to contain the co-factor SAM and the substrate n-terminal histidine, which is in agreement with its NHMT activity. The seven transmembrane helices (M1-M7) previously predicted with MEMSAT (FIG. 3A) were also predicted by the AlphaFold2 model (FIG. 3B). The topological orientation of the NHMT seven helices form a compact core, with hydrophobic residues facing outwards as one would expect for the membrane embedded domain with an approximate width of 40 Å, indicative of complete membrane spanning (FIG. 3B). Searching the AlphaFold2 predicted 7TM domain model against the PDB25 DALI database (Holm 2022) resulted in only weak hits with no 7TM proteins among them. The structures of these proteins were investigated manually and none of them had 7 helices. Although G protein-coupled receptors are typically synonymous with a 7TM domain, the helical organization of the 7TM helices in NHMT show no structural or sequence similarity to the GPCRs.

3.2 mRFP Fluorescence Tag Fused to N- or C-Terminal of AN4663

To determine the subcellular localization of AN4663, constructs of the AN4663 protein with an mRFP fluorescence tag fused to the N- or C-terminal of AN4663 were expressed in the knockout strain (NID2713), using a different integration site and promoter compared to the endogenous version of the NHMT gene, resulting in A. nidulans derivatives (NID2789 and NID2794). The strains were engineered using a CRISPR Cas9 plasmid targeting IS5 (a gene insertion site next to the gene AN7753 in A. nidulans (Nødvig et al 2018). The insertion region consists of two 1 kb homologous regions up-stream and down-stream from the CRISPR double stranded break in IS5. A promoter Ptef; coding sequences for either N- or C-terminal tagged AN4663; and a terminator Ttef were located between the two homologous regions. Additionally the two homologous regions were flanked by two SwaI sites to linearize the plasmid.

PRM analysis of A. nidulans expressing the AN4663-N-terminal or C-terminal mRFP fusion constructs revealed that methylation of LPMOs was stoichiometric for the N-terminally tagged version, suggesting it is functional and localized correctly in the cells (FIG. 2B). Conversely, the C-terminal mRFP tagged version was unable to methylate the LPMOs (see FIG. 2C). It is speculated that the C-terminal part of the NHMT is important for either the catalytic function or perhaps for the substrate recognition.

### 3.3 Catalytic Site Knock-Out

The alignment and structure prediction enabled identification of a conserved glutamate at position 340 within the SAM motif which is required for the catalytic activity of methyltransferases (Struck et al 2012). Site-directed mutagenesis of this glutamate to an alanine (E340A) lead to a loss of N-terminal histidine methylation generating a similar methylation phenotype to a complete knockout of AN4663 (FIGS. 2A, B, and C).

Collectively, these data show that AN4663 is the specific and sole NHMT in A. nidulans responsible for all N-terminal histidine methylation.

3.4 in Silico Analysis of NHMT Agrees with the Assignment of Catalytic Activity

Automated annotation of the AN4663 gene product suggests that it belongs to the spermidine synthase family of proteins (Wortman et al 2009). This is a subclass of the aminopropyl transferase family that catalyse the reaction between decarboxylated SAM and putrescine to form spermidine or longer polyamines. To distinguish AN4663 as a methyl transferase and not a spermidine synthase, the amino acid sequence of the soluble catalytic domain of AN4663 was BLAST searched against the SwissProt database of manually curated sequences. This resulted in 48 protein sequences with predicted spermidine synthase and methyl transferase activities including histamine methyl transferases that similarly methylate the nitrogen atom of imidazole rings. Multiple alignments of these sequences displayed strong conservation at the SAM binding positions including the SAM binding glutamine 340 within AN4663 that enabled us to generate a catalytic dead enzyme (as described in example 3.3). However, the alignment also revealed a single amino acid variance within a conserved motif that separates spermidine synthases from other methyl transferases. Specifically, spermidine synthases contain either an aspartic acid (D) or glutamic acid (E) in the G×G(D/E)G motif located within the SAM binding and catalytic pocket (FIG. 2D) that is required for spermidine synthase activity. However, isoleucine at residue position 322 (I322) replaces the D or E in this motif within AN4663 (FIG. 2D). Other methyl transferases similarly lack a D or E in the motif, separating them from spermidine synthases in our analysis.

### Example 4: Recombinant N-Terminal Histidine Methylation of LPMO LsAA9A by AN4663 in Yeast K. phaffii

The identified methyltransferase candidate AN4663 [SEQ ID NO.: 2] was co-expressed in the yeast Komagataella phaffii together with an AA9A LPMO from Lentinus similis (LsAA9A) [SEQ ID NO.: 15] to test for recombinant N-terminal histidine methylation (FIG. 4).

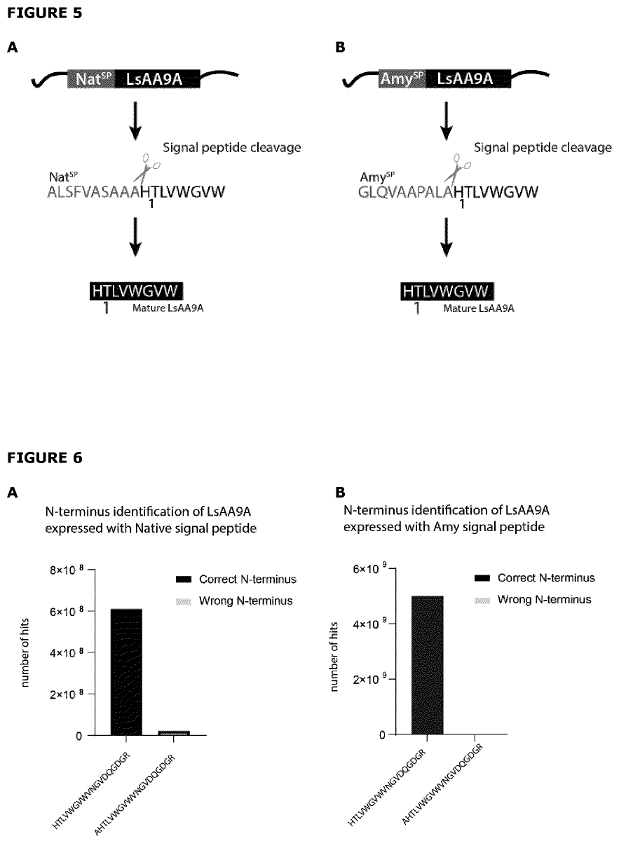

When producing a functional LPMO, the processing of the catalytically active N-terminal histidine amino acid via proper cleavage of the signal peptide is critical (Gaber et al 2020). Both the native signal peptide [SEQ ID NO.: 30] and AmySP from A. niger [SEQ ID NO.: 34] were tested for the secretion of LsAA9A. Prior to the experiment we confirmed by LC-MS/MS that the signal peptides for the secretion of LsAA9A were correctly processed (FIGS. 5&6). To express AN4663, an Episomal plasmid for stable propagation in K. phaffii was used. The NHMT protein sequence was kept intact, hoping that the transmembrane NHMT protein would behave similarly as in A. nidulans and follow the same transmembrane insertion pathway in the yeast K. phaffii.

4.1 Komagataella phaffii Engineering

K. phaffii was used as host cell (his4) (Thermo Fisher Scientific, Waltham, MA, USA). For co-expression, LsAA9A [SEQ ID NO.: 14] was cloned in the pLyGo plasmid to produce pLyGo-Kp-Native^{SP}-LsAA9A [SEQ ID NO.: 50] as well as pLyGo-Kp-Amy^{SP}-LsAA9A [SEQ ID NO.: 51](Hernandez Rollan et al 2021). The plasmids were linearized and transformed into his4 strain. Correct strains were validated by their ability to grow on histidine autotrophic media, and by colony PCR. All strains generated for the co-expression experiment are listed in supplementary information Table 2.

The DNA sequence of AN4663 was synthetized without introns [SEQ ID NO.: 47](IDT, Coralville, IA, USA), and cloned into an Episomal vector, using uracil excision cloning as described in Cavaleiro et al 2015.

Strains containing the genomic integrated LsAA9A were prepared electrocompetent for transformation with an episomal plasmid carrying the NHMT:

### 4.2 Co-Expression of LsAA9A LPMO and AN4663 Candidate

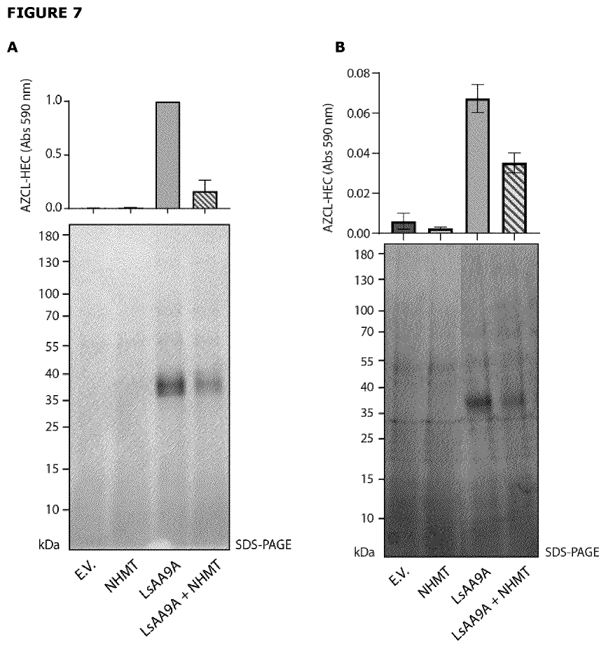

Single colonies of the transformed strain were used to inoculated 100 ml of BMGY medium (Pichia Expression Kit, Life Technologies, Carlsbad, CA, USA) and grown at 28° C. with shaking at 250 rpm for two days. From the saturated culture, a starting culture of OD600 1.0 was prepared in 200 mL of Buffered Methanol Complex (BMMY) medium with Zeocin (100 μg/mL) when needed using a baffled flask and the cultures placed at 28° C. with shaking at 250. Expression was maintained with the addition of 1% methanol to all the cultures for an additional four days. OD measurements were carried out daily and the cultures were plated on zeocin containing plates at the end of the expression study to confirm for the presence of the episomal vector. On the last day, the cells were collected by centrifugation at 5000 g for 20 minutes and the supernatants were subjected to filter sterilization. Secretion of LsAA9A was confirmed by SDS-PAGE (FIG. 7).

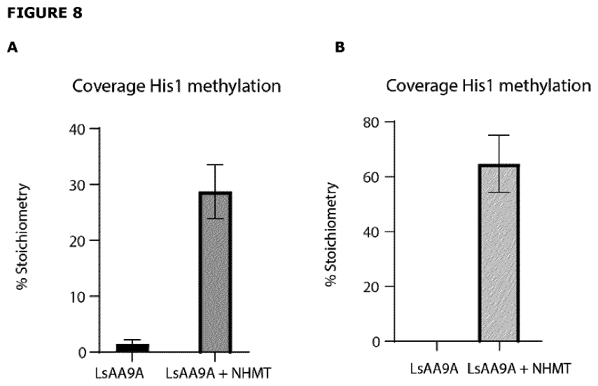

Samples were then analyzed by a targeted proteomic approach to identify the N-terminal methylation of LsAA9A. N-terminal histidine methylation of LsAA9A was only identified when AN4663 was present (FIG. 8). The N-terminal methylated and unmethylated peptides were quantified, showing a methylation stoichiometry on average of 65% of the peptides for the Amy signal peptide (FIG. 8B), and 29% for the native signal peptide (FIG. 8A). These results demonstrate that is possible to engineer K. phaffii as a genetically-modified host to efficiently modify the N-terminal histidine of LPMOs by methylation.

### 4.3. Purification of Expressed LsAA9A

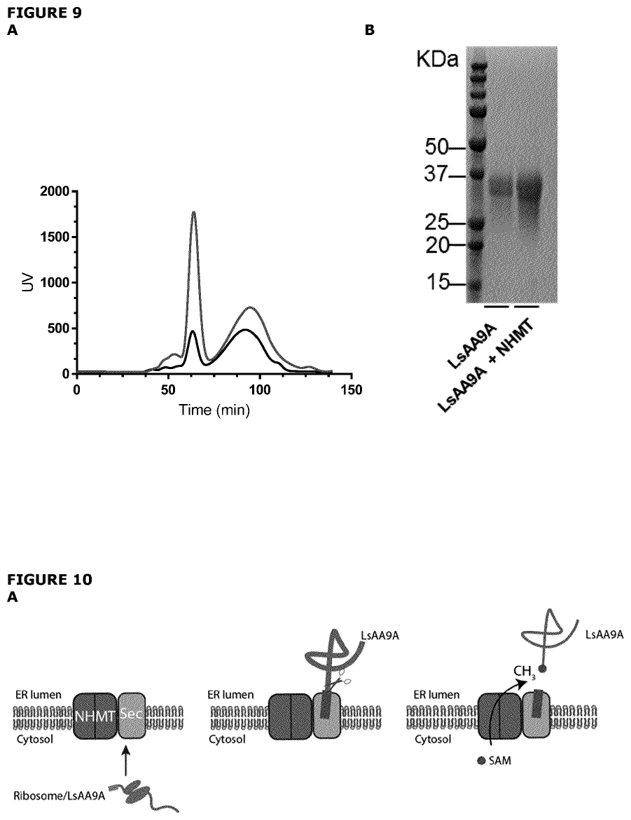

The supernatant of the K. phaffii cells co-expressing LsAA9A and the methyl transferase (NHMT) was buffer exchanged into 20 mM Bis-tris pH 5.9 using a 200 mL Sephadex G-25 column (Cytiva). Subsequently, the sample was applied to a 5 mL Q-Sepharose column and eluted with a gradient from 0-0.5M NaCl. Presence of protein was confirmed using precast criterion XT SDS-PAGE gels (BioRad) as shown in FIG. 9, and fractions containing protein were pooled. The relevant pools where concentrated to 5 mL using a vivaspin protein concentrator spin column with a 10MWCO filter (Cytiva). Subsequently the sample was cleaned with a 120 mL HiLoad Superdex 75 pg column (Cytiva). Fractions containing protein were pooled and analyzed using full acid hydrolysis and subsequent HPLC based quantification of individual amino acids.

### 4.4 Active Expression of LsAA9A

The activity of the LsA9A protein secreted by K. phaffii was confirmed using AZCL-HEC substrate, see FIG. 7 (Sigma-Aldrich, Saint Louis, MO, USA). 100 μL of the sample was mixed with 400 μL of the AZCL-HEC reaction mix (1 mg/mL AZCL-HEC substrate, 1 mM ascorbic acid, 100 μM Copper sulphate, and the volume adjusted with 100 mM Sodium acetate (pH 5)) and incubated at 50° C. with 1500 RPM shaking for one hour. Afterwards, the samples were centrifuged to get rid of the AZCL-HEC substrate and the absorbance measured at 590 nm.

### Example 5: Interaction at the ER Membrane Between the NHMT and the LPMO

As demonstrated above, the proper processing of the signal peptide, where the LPMO displays the N-terminal histidine amino acid, is vital for the NHMT to transfer the methyl group onto the LPMO. This suggests that the methylation happens during the secretory pathway in the endothelium reticulum (ER).

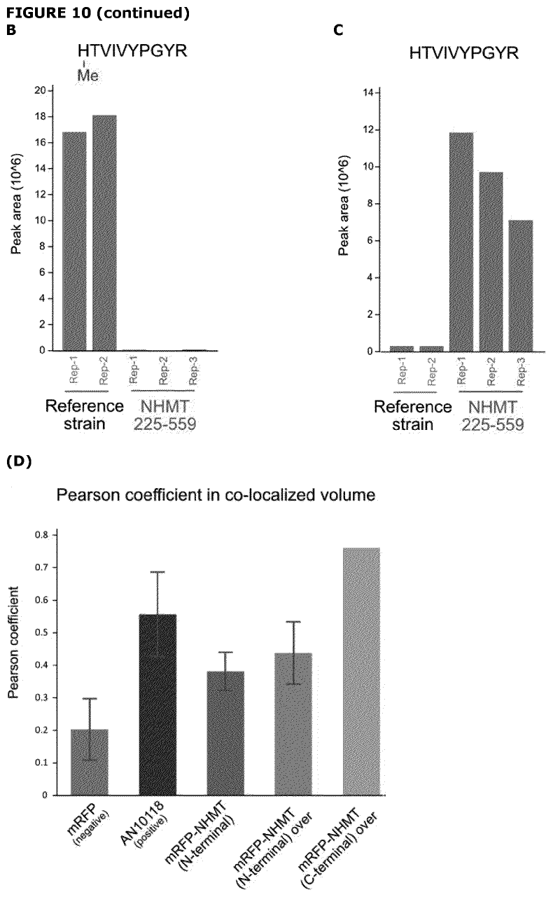

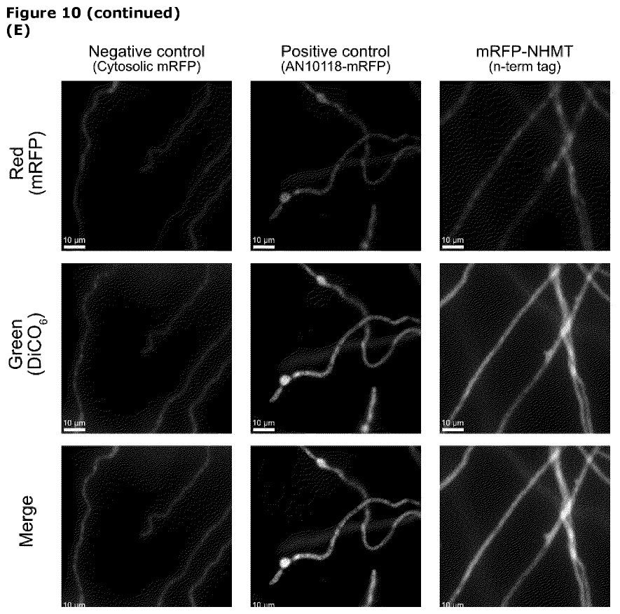

AN4663 is predicted to reside in the membrane of the endoplasmic reticulum and methylates N-terminal histidines of secreted proteins, as illustrated FIG. 10A.

A truncated variant of AN4663 lacking the transmembrane region (positions 1-224) was generated; the expression of this variant abolished the N-terminal histidine methylation capacity of the enzyme, indicating that the transmembrane region is critical for its specific activity (FIGS. 10B and 10C).

Further, co-localization cellular imaging was performed by fluorescence microscopy with the addition of an mRFP tag to the C- or N-terminal of full length NHMT alongside a positive ER marker, the fluorescent organelle probe DiOC₆: Fresh A. nidulans spores suspensions (10 μL of ˜10\hat{ }5 spores per mL) were inoculated on glass slides with 0.5 mL solid MM (as described above) with 4 mM L-arginine and incubated for 20 hours in petri dishes in micro-perforated bags at 37° C. A cover slide and immersion oil were applied and the ER was labelled with 10 μM 3,3′-Dihexyloxacarbocyanine iodide (DiOC6) for 20 minutes. Images of A. nidulans were acquired with a Leica DMI6000 widefield microscope, equipped with an 63×1.40 OIL HC PL APO objective and operated with LAS X (version 3.3.3) software. Three images of mRFP tagged proteins, green mitochondrial membrane dye (DiOC6) and bright field were acquired for each sample. Collected images were processed using the Imaris software version 9.8.2 (Bitplane AG, Zürich/Oxford Instruments, Abingdon, Oxfordshire, England). Background subtraction was performed followed by manual segmentation of A. nidulans filaments and saved as surfaces for each image and co-localization of mRFP and DiOC6 labelled components were determined using Imaris Coloc tool. Intensity threshold of 1000 and 600 were used respectively for the mRFP (red) and DiOC6 (green) channels in all images.

Cytosolic mRFP was used as a negative ER membrane control, and a mannosyltransferase protein known to localize to the ER (AN10118, UniProt entry C8VRA6) involved in protein glycosylation was tagged with mRFP and used as a positive ER localization control. The imaging analysis clearly shows co-localization of all mRFP-tagged AN4663 NHMT protein with the ER membrane control RFP protein, rather than with the cytoplasmic RFP, confirming the subcellular localization of the tagged AN4663 NHMT in the ER (FIGS. 10D and 10E).

### Example 6: Recombinant N-Terminal Histidine Methylation of GLP-1 by AN4663 in Yeast K. phaffii

The yeast species K. phaffii and S. cerevisiae are genetically engineered by integration into the genome of a gene [SEQ ID NO.: 55] encoding an AmySP-GLP-1-HSA fusion protein [SEQ ID NO.: 56] operably linked to the methanol-inducible promoter AOX [SEQ ID NO.: 10]; and by introduction of the gene encoding the N-terminal histidine methyltransferase protein (AN4663 [SEQ ID NO.: 2]).

The gene encoding the GLP-1-HSA is cloned in the pLyGo-Kp-2 plasmid down-stream of the AmySP coding sequence and the AOX promoter. The resulting recombinant expressing vector, pLyGo-Kp-2-AmySP-GLP-1-HSA, is linearized and inserted into the genome of K. phaffii and/or S. cerevisiae. The gene encoding methyltransferase is either integrated into the genome in the respective host cell or co-expressed using an episomal plasmid and expressed under the control of the strong constitutive promoter pGAP. The expression of the recombinant fusion AmySP-GLP1-HSA is induced upon induction with methanol for four days; then the supernatant is collected, and the GLP-1 histidine 1 amino acid is analyzed for methylation using proteomics.

K. phaffii cells comprising a gene encoding the fusion protein comprising a—mating factor SP-GLP-1-HAS are reported to express and secrete the GLP-1-HAS fusion protein (Dou et al., 2008). The precursor AmySP-GLP-1-HAS expressed by the host cells is predicted to processed during expression to yield a GLP-1 peptide having an N-terminal histidine residue (according to SignalP-6.0 [https://services.healthtech.dtu.dk/service.php?SignalP-6.0]).

### Example 7: NHMT Protein Production and In Vitro N-Terminal Histidine Methylation

A synthetic DNA encoding AN4663 was obtained without introns as a gBlock codon optimized (IDT, Coralville, IA, USA), and the region corresponding to the cytosolic domain (residues 220-558) as predicted by PSIPRED software (McGuffin et al 2000) (FIG. 3), was PCR amplified with SapI containing primers for LyGo cloning into the LyGo-Ec-1 vector as described in (Hernandez-Rollan et al., 2021). The resulting plasmid was transformed into E. coli BL21.

E. coli BL21 (DE3) cells transformed with pLyGo-Ec-1-Cytosolic AN4663 were used to produce the cytosolic domain. For expression, a single colony was inoculated in 25 mL of LB medium supplemented with kanamycin and placed at 37° C., 250 rpm. The day after, the culture was diluted 1:100 in 500 mL LB with kanamycin until an OD600 of 0.3, at that point the culture was transferred to an incubator at 18° C., 180 rpm. After recovery at 18 degrees for 1 hour, the culture was induced with 1 mM IPTG for 20 hours. Cultures were harvested by centrifugation at 8000 g, 4° C. for 20 minutes. The pellets were frozen at −80° C. until cell lysis. Whole cell lysis was performed by thawing—freezing cycles as described in (Hernandez-Rollan et al., 2021). Only the soluble fraction was used for Ni-NTA purification using Ni-NTA Superflow (Qiagen, Hilden, Germany). Purification, TEV cleavage, and reverse IMAC were followed as described in (Hernandez-Rollan et al., 2021).

In vitro N-terminal histidine methylation of a protein or peptide by the purified histidine N-methyltransferase enzyme or portion of the enzyme containing, but not limited to, only the catalytic histidine N-methyltransferase domain, or the catalytic histidine N-methyltransferase domain fused to other protein or biomolecules i.e. to increase stability, is performed by incubating the enzyme with a donor S-Adenosyl methionine containing, but not limited to, a methyl group that is to be transferred to the target substrate such as, but not limited to, peptide, protein, incretin hormone containing a n-terminal histidine amino acid residue.

The reaction can be optimized to reach highest stoichiometric methylation of target substrate by optimizing a large number of reaction conditions such as, but not limited to, varying the concentration of the histidine N-methyltransferase enzyme, the substrate concentration, the donor S-Adenosyl methionine containing, but not limited to, a methyl group, varying the concentration of the target substrate, varying the concentration of the reaction volume, and buffer concentration such as, but not limited to, tris(hydroxymethyl)aminomethane (commonly referred to as Tris), 4-(2-hydroxyethyl)-1-piperazineethanesulfonic acid (commonly referred to as HEPES), the pH of the reaction solution, the temperature at which the reaction is carried out, and the time duration of the reaction. N-methyltransferase enzyme kinetics can be further optimized by the addition of co-factors such as, but not limited to, adenosine triphosphate (ATP), guanosine diphosphate (GDP), guanosine triphosphate (GTP), nicotinamide adenine dinucleotide (NAD), and nicotinamide adenine dinucleotide+hydrogen (NADH or NAD+). Additional individual chemical and or biological components can be added to increase reaction efficiency such as, but not limited to, calcium chloride, magnesium chloride, and sodium chloride.

The reaction efficiency can be monitored using different methods and techniques including, but not limited to, measuring the mass of the substrate using a mass spectrometer, utilizing an antibody specific for methylated histidine's, measuring the accumulation of S-Adenosyl-L-homocysteine, or other indirect measurements based on fluorescence, light, or radioactivity.

### Example 8: Conservation of AN4663

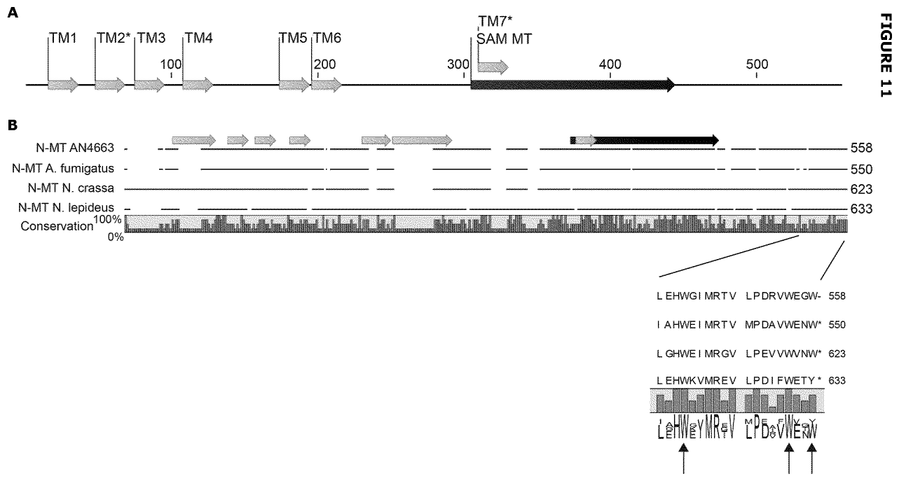

BlastP analysis of the AN4663 protein sequence was performed in the MycoCosm genome portal database at JGI (Grigoriev et al., 2012). This analysis revealed that filamentous fungi within ascomycota and basidiomycota had an ortholog and no paralogs to this protein. To illustrate this conservation of AN4663 with orthologs in the kingdom, firstly, the structural features of the AN4663 protein was dissected by TMD prediction in TOPCONS2 database (Tsirigos et al., 2015) and a conserved domain prediction in CCD v3.19, NCBI (Shennan Lu et al. 2021) as seen in FIG. 11A, where the relative positions of predicted TMDs and catalytic SAM-dependent methyl transferase domain are displayed in the AN4663 protein sequence and visualized in CLC Main Workbench v21 (Qiagen, Hilden, Germany). This predicted features of AN4663 was compared to three selected orthologs picked up from the BlastP analysis in Mycocosm in an alignment using default alignment settings in CLC Main Workbench v21 as seen in FIG. 11B. The three orthologs aligned to AN4663 were chosen from fungal species that through their life style were expected or known to produce LPMO AA9s, and also represented a member of the same genus (Aspergillus fumigatus), and a highly diverse ascomycete (Neurospora crassa), and a basidiomycete (Neolentinus lepideus). The region spanning the TMDs (N-terminal) revealed to be less stringent in conservation overall for the four species, whereas the catalytic domain in the C-terminus of the protein showed the highest conservation. The bottom panel of FIG. 11B showed that conserved residues are distributed throughout the sequence alignment, but especially the N-terminal part of the protein showed larger differences for the phylogenetically more distant species. As an example of key residues, a zoom in on the sequence in the very C-terminus revealed conserved Tryptophan/tyrosine residues, which is proposed to be important for the functionality of the protein.

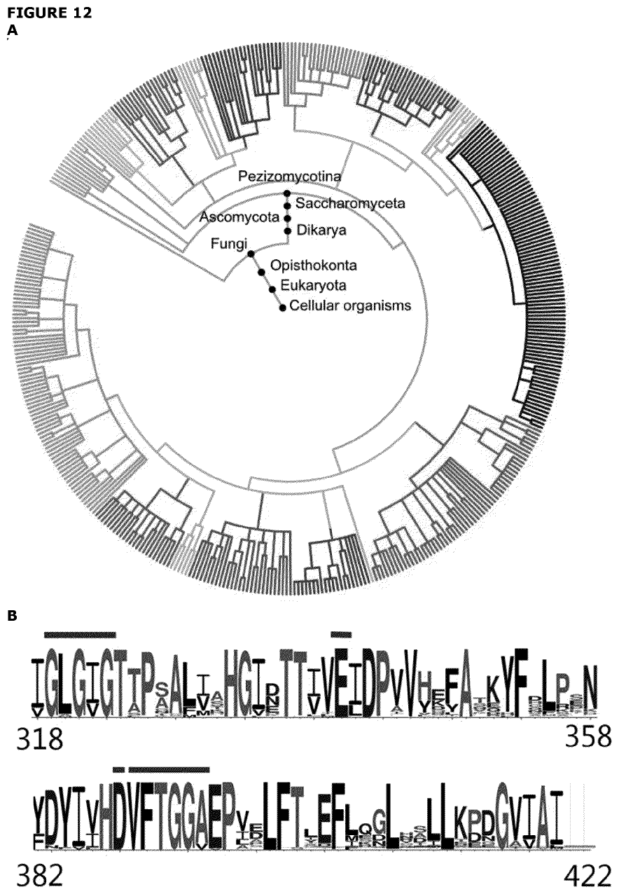

UniProt BLAST search of the 7TM domain (aa residues 1-215) of AN4663 (used as input for a pBLAST search against the uniprotkb_refprotswissprot database using the BLOSUM-45 matrix and an exp cutoff at 1e-4) resulted in 452 matches to homologous 7TM domains containing proteins exclusively to organisms within the Ascomycota subphylum pezizomycotina (with the exception of one unclassified fungal organism), which contains the filamentous Ascomycetes including Aspergillus (FIG. 12A). The analysis revealed that ˜97% of the matches are single copy genes within their respective organisms. Moreover, all 452 transmembrane domain sequences were linked to soluble domains.

Alignment of the soluble domains linked to the 452 transmembrane domain sequences by MUSCLE v. 3.8; visualized as sequence motifs in WebLogo 3.0 (FIG. 12B) showed that within the region 318-358, the methyl transferase motif at 319-323 as well as the SAM binding glutamate in position 340 are present in all sequences. The amino acid positions are numbered relative to AN4663. Further, it was found that all the soluble domains contain a highly conserved region 389-394, uniquely found in proteins with the NHMT 7TM domain (FIG. 12B). This analysis thus further confirmed that the NHMT represents a hitherto uncharacterized protein family containing a 7TM domain linked to a methyl transferase domain that methylates n-terminal histidine in filamentous fungi.

### Example 9: Stability of the Methylated LPMO

To assess the impact of H2O2 on LsAA9a and LsAA9A+NHMT, 200 μL reactions were prepared with 20 mM MES, pH 6.6, 1 μM enzyme, 0.5 μM CuCl2, 500 μM cellopentaose, 100 μM ascorbate and 0, 100, 300 and 500 μM H2O2. Components were added in the listed order and 1 hour equilibration delay was introduced before addition of ascorbate. Reactions were incubated 30 min at 25° C. and stopped by addition of NaOH to 0.1 M and analysed by HPAEC-PAD according to Westereng et al. (2013).

### REFERENCES

Blum, M. et al. The InterPro protein families and domains database: 20 years on. Nucleic Acids Res. 49, D344-D354 (2021).
Cavaleiro, A. M., Kim, S. H., Seppala, S., Nielsen, M. T. & Nørholm, M. H. H. Accurate DNA Assembly and Genome Engineering with Optimized Uracil Excision Cloning. ACS Synth. Biol. 4, 1042-1046 (2015).
Cove, D. J. The induction and repression of nitrate reductase in the fungus Aspergillus nidulans. Biochim. Biophys. Acta—Enzymol. Biol. Oxid. 113, 51-56 (1966).
Dou W-F et al., Expression, purification, and characterization of recombinant human serum albumin fusion protein with two human glucagon-like peptide-1 mutants in Pichia pastoris Protein Expression and Purification Volume 61(1), 45-49 (2008).
Gaber, Y. et al. Heterologous expression of lytic polysaccharide monooxygenases (LPMOs). Biotechnol. Adv. 43, 107583 (2020).
Grigoriev, I. V. et al. The Genome Portal of the Department of Energy Joint Genome Institute. Nucleic Acids Res. 40, D26-32 (2012).
Hartmuth et al 2011. Click Chemistry: Diverse chemical function from a few good reactions. Angewandte Chemie. Vol 40, Issue 11, pp 2004-2021.
Hernández-Rollán, C. et al. LyGo: A Platform for Rapid Screening of Lytic Polysaccharide Monooxygenase Production. ACS Synth. Biol. 10, 897-906 (2021).
Holm, Dali server: structural unification of protein families. Nucleic Acids Research. 50, W210-W215 (2022).
Letunic et al, Interactive Tree Of Life (iTOL) v5: an online tool for phylogenetic tree display and annotation. Nucleic Acids Research. 49, W293-W296 (2021).
Lu, S. et al. CDD/SPARCLE: the conserved domain database in 2020. Nucleic Acids Res. 48, 265-268 (2020).
McGuffin, L. J., Bryson, K. & Jones, D. T. The PSIPRED protein structure prediction server. Bioinformatics 16, 404-405 (2000).
Mistry, J. et al. Pfam: The protein families database in 2021. Nucleic Acids Res. 49, D412-D419 (2021).
Nielsen, M. L., Albertsen, L., Lettier, G., Nielsen, J. B. & Mortensen, U. H. Efficient PCR-based gene targeting with a recyclable marker for Aspergillus nidulans. Fungal Genet. Biol. 43, 54-64 (2006).
Nødvig, C. S., Nielsen, J. B., Kogle, M. E. & Mortensen, U. H. A CRISPR-Cas9 System for Genetic Engineering of Filamentous Fungi. PLoS One 10, e0133085 (2015).
Nødvig, C. S. et al. Efficient oligo nucleotide mediated CRISPR-Cas9 gene editing in Aspergilli. Fungal Genet. Biol. 115, 78-89 (2018).
Shennan Lu et al. (2020), “CDD/SPARCLE: the conserved domain database in 2020.”, Nucleic Acids Res. 48(D1)265-8.
Sigrist, C. J. A. et al. New and continuing developments at PROSITE. Nucleic Acids Res. 41, D344-D347 (2012).
Struck, A. W., Thompson, M. L., Wong, L. S. & Micklefield, J. S-Adenosyl-Methionine-Dependent Methyltransferases: Highly Versatile Enzymes in Biocatalysis, Biosynthesis and Other Biotechnological Applications. ChemBioChem 13, 2642-2655 (2012).
Torlak, E., Vaziri, M. & Dolby, J. MemSAT. in Proceedings of the 2010 ACM SIGPLAN conference on Programming language design and implementation—PLDI '10 341 (ACM Press, 2010). doi:10.1145/1806596.1806635.
Tsirigos, K. D. et al. The TOPCONS web server for combined membrane protein topology and signal peptide prediction. Nucleic Acids Research 43, W401-W407 (2015).
Westereng et al 2013. Efficient separation of oxidized cello-oligosaccharides generated by cellulose degrading lytic polysaccharide monooxygenases. Journal of Chromatography A. Volume 1271, Issue 1, 4 Jan. 2013, Pages 144-152.
Wortman et al, The 2008 update of the Aspergillus nidulans genome annotation: a community effort. Fungal Genet Biol. 46 Suppl 1, S2-13 (2009).

1. A genetically-modified cell for production of a target peptide or polypeptide having a methylated N-terminal histidine residue, wherein the cell comprises:
a. a first gene comprising a first nucleic acid sequence encoding a first polypeptide exhibiting S-adenosylmethionine-dependent N-terminal histidine methyltransferase (NHMT) activity,
wherein the first gene is genetically engineered,
wherein the first polypeptide (i) is of fungal origin, (ii) is not native to said cell, (iii) comprises an N-terminal 7 transmembrane spanning domain, and (iv) comprises a soluble C-terminal NHMT catalytic domain, wherein the soluble C-terminal NHMT catalytic domain comprises a SAM binding domain having a glutamate residue, and
b. a second gene comprising a second nucleic acid sequence encoding a target peptide or polypeptide, or a precursor thereof,
wherein the target peptide or polypeptide comprises a N-terminal histidine residue; and
wherein the first polypeptide facilitates methylation of the N-terminal histidine residue of the target peptide or polypeptide.

2. The genetically-modified cell according to claim 1, wherein the SAM binding domain comprises an amino acid sequence having at least 60% sequence identity to amino acid residues 289 to 502 of SEQ ID NO.: 2.

3. The genetically-modified cell according to claim 2, wherein the first polypeptide comprises a GXGX′G motif at positions corresponding to amino acid residues 319-323 of SEQ ID NO.: 2, wherein the position X in the GXGX′G motif corresponding to amino acid residue 320 of SEQ ID NO.: 2 is any amino acid and the position X′ in the GXGX′G motif corresponding to amino acid residue 322 of SEQ ID NO.: 2 is any amino acid other than D or E.

4. The genetically-modified cell according to claim 2, wherein the first polypeptide comprises a VFTGG motif at positions corresponding to amino acid residues 389-394 of SEQ ID NO.: 2.

5. The genetically-modified cell according to claim 1, wherein the first polypeptide comprises an amino acid sequence having at least 60% sequence identity to amino acid residues 220 to 558 of SEQ ID NO.: 2.

6. The genetically-modified cell according to claim 1, wherein the first polypeptide consists of an amino acid sequence having at least 60% sequence identity to SEQ ID NO.: 2.

7. The genetically-modified cell according to claim 1, wherein the second gene comprising a second nucleic acid sequence encoding the target peptide or polypeptide is genetically engineered and not native to the genetically-modified cell.

8. The genetically-modified cell according to claim 1, wherein the precursor of the target peptide or polypeptide is a secreted peptide or polypeptide having a pre- or a prepro-domain, and wherein the pre- or prepro-domain is removed prior to methylation of the N-terminal histidine residue of the target peptide or polypeptide.

9. The genetically-modified cell according to claim 1, wherein:
a. the target polypeptide is a lytic polysaccharide monooxygenase, and
b. the target peptide is an incretin hormone.

10. The genetically-modified cell according to claim 1, wherein the second nucleic acid sequence encodes a lytic polysaccharide monooxygenase having an any signal peptide in substitution for a native signal peptide.

11. The genetically-modified cell according claim 9, wherein the incretin hormone is glucagon-like peptide-1 (GLP-1) or an analogue thereof, wherein the GLP-1 or the analogue is selected from the group having an amino acid sequence of SEQ ID NO.: 37, 39, 41, 43, 45, and 46.

12. The genetically-modified cell according to claim 1, wherein the genetically-modified cell is selected from K. phaffii and a species of Saccharomyces.

13. A method for production of a target peptide or polypeptide having a methylated N-terminal histidine residue, comprising the steps of:
c. providing a genetically-modified cell according to claim 1, or a cell population derived therefrom;
d. culturing the cell or cell population under conditions allowing expression of the first gene comprising the first nucleic acid sequence encoding the first polypeptide exhibiting N-terminal histidine methyltransferase activity and the second gene comprising the second nucleic acid sequence encoding the target peptide or polypeptide, or the precursor thereof,
e. recovering the target peptide or polypeptide having a methylated N-terminal histidine residue.

14. The method according to claim 13, wherein the target polypeptide or precursor thereof is a lytic polysaccharide monooxygenase.

15. The method according to claim 13, wherein the target peptide or precursor thereof is an incretin hormone.

16. Use of the genetically-modified cell according to claim 1 for production of a target peptide or polypeptide having a methylated N-terminal histidine residue.

17. The use according to claim 16, wherein
a. the target polypeptide is a lytic polysaccharide monooxygenase, and
b. the target peptide is an incretin hormone.
